(19) United States
(12) Patent Application Publication
(10) Pub. No.: US 2011/0215273 A1
(43) Pub. Date: Sep. 8, 2011

(54) Hydrofluoroolefins, manufacture of hydrofluoroolefins and methods of using hydrofluoroolefins

(75) Inventors: Ercan Uenveren, Hannover (DE); Johannes Eicher, Sehnde (DE); Wolfgang Kalbreyer, Steyerberg (DE)

(73) Assignee: SOLVAY FLUOR GMBH, Hannover (DE)

(21) Appl. No.: 13/128,594

(22) PCT Filed: Nov. 13, 2009
(86) PCT No.: PCT/EP2009/065175

§ 371 Date: May 10, 2011

Related U.S. Application Data
(60) Provisional application No. 61/114,320, filed on Nov. 13, 2008.

(51) Int. Cl. C09K 5/00; C07C 17/25; C07C 21/18; C08J 9/04; C09K 3/00

(52) U.S. Cl. 252/78.1; 570/155; 570/156; 570/136; 521/116; 252/182.15; 252/182.12

(57) ABSTRACT

A hydrofluoroolefin and hydrofluoroolefin isomers and a process for manufacture them comprising eliminating HF from a fluorinated precursor compound are described. The fluorinated precursor compound may be provided by fluorinating a chlorinated precursor. The fluorinated precursor compound may be a fluorinated alkane. The hydroolefines are suitable as blowing agents, heat transfer fluids, or drying agents or degreasing solvents.

The invention concerns the manufacture of hydrofluoroolefins and uses of the hydrofluoroolefins obtained.

Apparatus for heating and cooling today are often operated with saturated hydrofluorocarbon compounds, for example with HFC-134a (1,1,1,2-tetrafluoroethane). Saturated hydrofluorocarbons are also applied for the manufacture of foamed plastics, e.g. for the manufacture of polystyrene foam (“XPS”), polyurethane (“PUR”) or polyisocyanurate (“PIR”) foams. Foams have a widespread commercial use in a variety of different applications. Saturated hydrofluorocarbons are also applied for other purposes, e.g. as solvent, for cleaning operations such as degreasing or for heat transfer.

The hydrofluorocarbons have no detrimental influence on the stratospheric ozone, there are concerns due to their contribution to the greenhouse effect; i.e., they contribute to global warming.

WO 2007/053674 discloses methods for making foams using blowing agents comprising unsaturated fluorocarbons; a significant number of different unsaturated hydrofluorocarbons is disclosed as suitable. The preferred unsaturated hydrofluorocarbons are those of formula R¹CH═CHR² wherein R¹ and R² are, independently, C₁ to C₆ perfluoroalkyl groups.

WO 2004/096737 describes new fluorobutenes.

WO 2009/010472 discloses the preparation of halogen and hydrogen containing alkenes over metal fluoride catalysts with a high specific surface and high Lewis acidity.

In view of the foregoing, there is a continuing need for hydrofluoroolefins, processes for their manufacture and their use.

These and other objects of the present invention are achieved by the present invention as outlined in the claims.

One aspect of the present invention concerns a process for the manufacture of hydrofluoroolefines.

The manufacture process is carried out by (a) providing a chlorinated precursor compound; (b) fluorinating said chlorinated precursor to provide a fluorinated precursor compound; (c) eliminating HF from said fluorinated precursor compound to form at least one hydrofluoroolefin.

In particular, step (c) is also a separate object of the invention.

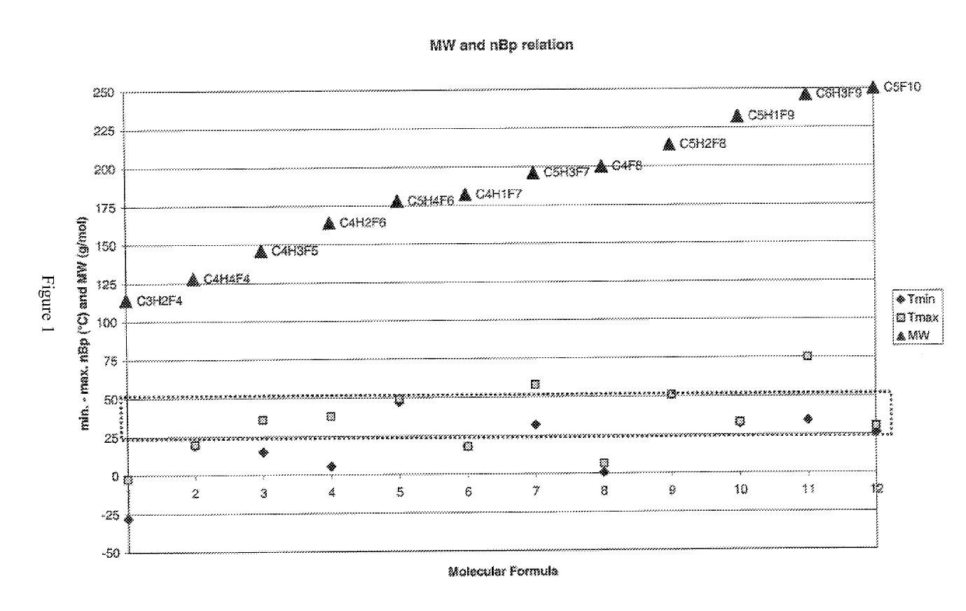

FIG. 1 shows the molecular weight (“MW”) for certain hydrofluoroolefins. FIG. 1 also indicates the lowest boiling point (“Bp”) and the highest boiling point (as far as known) of isomers of the hydrofluoroalkenes with the respective formulas indicated in FIG. 1. For example, the hydrofluoroolefin of formula C₅H₁F₇ has an isomer with a boiling point of 32° C. and an isomer with a boiling point of 58° C. The lowest boiling point is Tmin, the highest boiling point is Tmax.

The chlorinated precursor compound in step (a) may be provided by a reaction of a chlorinated alkene (e.g., any alkene compound identified as ‘reactant 1’ in Table 1) with a chlorine-containing compound, such as Cl₂, CCl₄, CCl₃—CCl₃ (e.g., reactant 2 in Table 1), or by chlorination of chlorinated alkanes (e.g., any chlorinated alkane compound identified as ‘reactant 1’ in table 1. Examples of suitable chlorinated precursor compounds are shown in Table 1 and identified as ““Intermediate”. The chlorinated precursor compound may have at least 3 halogen atoms, or at least 5 halogen atoms, or from 3 to 11 halogen atoms, or from 5 to 11 halogen atoms, wherein the halogen atoms in the chlorinated precursor compound may include only chlorine atoms or a combination of fluorine and chlorine atoms.

The term “chlorinated alkene” preferably denotes compounds consisting of carbon, hydrogen and chlorine or carbon, hydrogen, chlorine and fluorine.

The chlorinated alkenes have at least 2 carbon atoms and are substituted by at least 1 chlorine atom and by at least 1 hydrogen atom; preferably, they are substituted by at least 1 chlorine atom and by at least 2 hydrogen atoms. Preferably, they have 2 to 5 carbon atoms.

Preferred chlorinated alkenes are those of formula (I)

R¹CH═CClR² (I)

wherein R¹ is H; a C₁ to C₃ alkyl group; or a C₁ to C₃ alkyl group which is substituted by at least 1 halogen atom selected from the group consisting of chlorine and fluorine; and R² is H; a C₁ to C₃ alkyl group; or a C₁ to C₃ alkyl group which is substituted by at least 1 halogen atom selected from the group consisting of chlorine and fluorine; preferably, the sum of the carbon atoms of R¹ and R² is an integer equal to or lower than 4.

Very preferred chlorinated alkenes of formula (I) are those wherein R¹ is H, CH₃ or CF₃. Very preferred chlorinated alkenes of formula (I) are those wherein R² is H, a C₁ or C₂ alkyl group or a C₁ or C₂ alkyl group which is substituted by at least 1 chlorine or fluorine atom. Especially preferred chlorinated alkenes of formula (I) are those wherein R¹ is H, CF₃ or CH₃, and R² is H, CH₃, CCl₃, CF₃ or CH₂CF₃. The chlorinated alkenes are known or can be manufactured from saturated alkenes by dehydrofluorination or dehydrochlorination as will be described in detail below. Some of the precursors are intermediates obtainable in fluorination reactions. For example, CH₂═C(Cl)CH₂CF₃ and CH₃C(Cl)═CH₂CF₃ are intermediates in the fluorination reaction of 1,1,1,3,3-pentachlorobutane with HF to form 1,1,1,3,3-pentafluorobutane.

The most preferred alkenes are those in table 1 in the column denoted as “Reactant 1”.

The term “chlorinated alkane” preferably denotes compounds consisting of carbon, hydrogen, chlorine and fluorine. Preferred chlorinated alkanes are those of formula (II), C₅HₐCl_{b}F₃, wherein a is 1 to 4 and b is 5 to 8 with the proviso that the sum of a+b is 9. These compounds may be prepared by the addition of CCl₄ to CH₂═C(Cl)CH₂CF₃ and, when b is 6, 7 or 8, subsequent chlorination.

The most preferred chlorinated alkanes are those in table 1 in the column denoted as “Reactant 1”.

As mentioned above, the chlorinated precursor compound in step (a) may be provided by a reaction of the chlorinated alkene, especially one of the chlorinated alkenes described above, with a chlorine-containing compound, such as Cl₂, CCl₄, CCl₃—CCl₃, or by the reaction of chlorinated alkanes with chlorine.

The fluorinating step (b) may comprise or may consist of catalytic hydrofluorination.

Preferably, the hydrofluorination is performed in the liquid phase. Suitable catalysts and reaction conditions for the chlorine-fluorine exchange reaction are well known to the expert. Suitable catalysts are preferably selected from the group of halides of antimony, titanium, tin, niobium and tantalum. Highly suitable are, for example, titanium (IV) halides, especially titanium tetrachloride, titanium tetrafluoride and titanium chloride fluorides, antimony pentachloride, antimony pentafluoride and antimony chloride fluorides, and tantalum pentachloride, tantalum pentafluoride and tantalum chloride fluorides. The ratio of HF and chlorine atoms is preferably equal to or greater than 1. A preferred range is 1 to 10. Reaction temperature and duration of the reaction are selected such that a good yield of the fluorinated alkane is achieved in reasonable time. Preferably, the reaction is performed at a temperature in the range of 20 to 200° C., more preferably 20 to 150° C., if desired, under pressure.

If desired, the fluorination reaction includes a step of non-catalytic fluorination and a step of catalytic fluorination.

The fluorinated alkane can be isolated in a known manner, e.g. by aqueous workup or by fractionated distillation.

The fluorinated precursor compound in step (b) preferably includes a fluorinated alkane. Examples of suitable fluorinated precursor compounds are shown in Table 1 and are identified as ““Fluorinated Alkane”.

The fluorinated precursor compound or fluorinated alkane may have at least 5 fluorine atoms, or from 5 to 11 fluorine atoms. In preferred embodiments, the fluorinated precursor compound or fluorinated alkane does not include a chlorine atom. Preferred fluorinated precursor compounds are those of formula (IIIa), (IIIb) and (IIIc)

R¹CH₂—CF₂—R² (IIIa)

R¹CHF—CF₂—R² (IIIb)

R¹CF₂—CF₂—R² (IIIc)

Wherein R¹ is H; F; a C₁ to C₃ alkyl group; a C₁ to C₃ alkyl group, which is substituted by at least 1 fluorine atom; and R² is H; a C₁ to C₃ alkyl group; a C₁ to C₃ alkyl group, substituted by at least 1 fluorine atom, with the proviso that the number of carbon atoms in the fluorinated precursor compounds of formulae (IIIa), (IIIb) and (IIIc) is an integer equal to or greater than 3, and the number of fluorine atoms is at least 4. Preferably, the number of carbon atoms is equal to or greater than 4. Preferably, the number of fluorine atoms is equal to or greater than 6. Preferably, R¹ is selected from F; CF₃; CF₃CH₂; CF₃CHF; and CF₃CF₂; and R² is preferably selected from the group consisting of H; CH₃; CH₂F; CHF₂; CF₃CH₂; CF₃CHF; and CF₃CF₂. The hydrofluoroolefins formed according to the present invention have at least 4 fluorine atoms. Preferably, they have equal to less than 10 fluorine atoms.

The hydrofluoroolefins formed according to the present invention may have at least 6 fluorine atoms, or from 6 to 10 fluorine atoms. The hydroolefins with at least 6 fluorine atoms are preferred.

Especially preferred hydrofluoroolefines are those of formula (IV).

CₐH_{b}F_{c} (IV)

Wherein a, b and c are integers, a is 4 to 8, b is 4 to 10 and c is (2a−b), and a+b+c are 2a. Preferably, a is 4 to 6, b is 1 to 4, and c is (2a−b). More preferably, a is 5 or 6, b is 1 to 4, and c is (2a−b). Examples of hydrofluoroolefins formed according to the present invention are shown in Tables 1, 2 & 3a-3i and are identified as ““Olefin” or “Alkene”.

The process according to the present invention may generate a hydrofluoroolefin with a single structure or may generate two or more hydrofluoroolefins having the same molecular formula (isomers).

These isomers may be structural isomers i.e., they have the same molecular formula but different connections between atoms (bonding), and/or stereoisomers, i.e., have the same molecular formula, the same connections between atoms, but different arrangements of the atoms in the three dimensional space. The stereoisomeric forms of the hydrofluoroolefins formed by such process may be defined using the E-Z notation. A molecule gets the “E” notation if the groups with highest priority are on the opposite side of a double bond. Examples of hydrofluoroolefin isomers formed according to the present invention are shown in Tables 1, 2 & 3a-3i and are identified as “alkene isomere”.

The hydrofluoroolefin and hydrofluoroolefin isomers may comprise the following non-limiting molecular formula:

| MF | MW |
|---|---|
| C₃H₂F₄ | 114 |
| C₄H₁F₇ | 182 |
| C₄H₂F₆ | 164 |
| C₄H₃F₅ | 146 |
| C₄H₄F₄ | 128 |
| C₅H₁F₉ | 232 |
| C₅H₂F₈ | 214 |
| C₅H₃F₇ | 196 |
| C₅H₄F₆ | 178 |
| C₆H₃F₉ | 246 |

Tables 1 & 2 illustrate various embodiments of the present invention, in which the process may employ various reactions (1-29) to generate various hydrofluoroolefins and hydrofluoroolefin isomers. For example, the hydrofluoroalkene obtainable in reaction 5 is C₄H₄F₄. Three isomers exist: (E)-CF₃—CH═C(F)CH₃ wherein the CF₃ group and the F atom are opposite to each other, (Z)—CF₃—CH═C(F)CH₃ and the isomer CF₃—CH₂—C(F)═CH₂.

The reactions 1-8 can for example be telomerization reactions. Generally, such reactions are catalyzed. Suitable catalysts are known.

WO 98/50329 discloses that Cu(I) and Cu(II) compounds are suitable catalysts. The copper compound may be an inorganic copper compound, or an organic copper compound. CuCl₂ is very suitable. Preferably, a co-catalyst is applied. Preferred co-catalysts are amines, especially isopropyl amine and tert-butyl amine. The reactions 1-8 can for example be telomerization reactions performed with CuCl₂ and tert-butyl amine (t-BuAm).

In these telomerization reactions additional solvent might be required but not necessarily. If the telomerization is performed in the presence of a solvent or solvent mixture, then the solvent is preferably selected from the group consisting of nitriles, dinitriles, amides and trialkyl phosphinoxides. N-Methyl pyrrolidone, N,N-dimethyl acetamide, tri-(n-hexyl)phosphinoxides, tri-(n-octyl)phosphinoxides, n-octyl-di-(n-hexyl)phosphinoxides, n-hexyl-di-(n-octyl)phosphinoxides and their mixtures are preferred solvents. It is especially preferred to apply the chloroalkane which is reactant in the telomerization process, as solvent. For example, when CCl₄ is added to unsaturated compounds, it is applied in excess and functions as reactant and as solvent.

These reactions 1-8 may also be carried out with CuCl₂ and triphenylphosphine (PPh₃) with sulfolane as a solvent.

The reactions 1-8 may also be carried out using Fe and phosphites as catalyst and co-catalyst, as disclosed in WO 2008/040803.

In a very preferred embodiment, the unsaturated starting compounds have a CH₂═Cl— group.

Reactions 9-29 are suitably photochlorination reactions, in other words, Chlorine addition and/or substitution reactions. Since photochlorination might not be selective, reaction mixtures could be obtained. However, by adjusting the chlorine concentration some products in the mixture might be favored.

The photochlorination reaction is preferably performed in the liquid phase, preferably in the absence of a solvent. A UV light emitting lamp or respective LEDs can be applied as UV source. Often, chlorine is bubbled continuously through the liquid compound which is to be chlorinated. The compound to be chlorinated is preferably deoxygenated by passing dry nitrogen through it. The temperature during chlorination is preferably kept between 0 and 80° C. Samples can be taken from the liquid to monitor the degree of chlorination. The amount of chlorine is adapted to the desired reaction: the more hydrogen atoms are to be substituted by chlorine, the higher the molar ratio of chlorine in respect to the compound to be chlorinated. After termination of the reaction, any chlorine and HCl are removed from the reaction mixture, e.g. by stripping with nitrogen. The chlorinated product can be purified by fractionated distillation or can be fluorinated without isolation. A suitable photo chlorination process is described in U.S. Pat. No. 5,705,779.

Especially if the chlorination reaction relates to the addition of chlorine to a double bond, as is the case in reactions 9-23, the reaction can also be promoted by other means, e.g. by free-radical initiators, or by certain metal salts. This is disclosed in WO 02/12153, for example on pages 3-10.

Although not shown, the final products of the reactions 9-23 might also be obtained via direct chlorination of PCBa (1,1,1,3,3-pentachlorobutane).

In Table 1, X stands for the total halogen number in the haloalkane molecule; C, H, F correspond to the number of carbon, hydrogen and fluorine atoms, respectively, in the hydrofluoroolefin (identified as “Olefin” or “Alkene”); F/H ratio corresponds to the fluorine-to-hydrogen ratio in the hydrofluoroolefin (identified as “Olefin” or “Alkene”).

MF in all Tables stands for molecule formulas of the hydrofluoroolefin.

MW in all Tables stands for molecular weight.

The total number of possible isomers (in the “isomers” column) and the expected structures of the final product (in the various “Alkene Isomer” columns) are also given in Table 1.

The hydrofluoroolefin structures shown in bold in Tables 1 and 2 are analog to the very toxic CF₂═CF₂ (TFE) and CF₂═CF—CF₃ (HFP) due to the CF₂′CF— functional group.

The perfluorinated olefins might possess higher global warming potential (GWP) values than the hydrofluoroolefins (HFO).

FIG. 1 represents the minimum-maximum boiling points (Tmin, Tmax) of the hydrofluoroolefins and some perfluorinated olefins of increasing molecular weight (MW). For a given number of carbon atoms, the various hydrofluoroolefins have a higher Tmin and Tmax than the perfluorinated olefin. Tmin indicates the boiling point of the isomer with the lowest boiling point (as far as known) and Tmax indicates the boiling point of the isomer with the highest boiling point (as far as known).

| | Tmin | Tmax | |
|---|---|---|---|
| MF | (° C.) | (° C.) | MW |
| C₃H₂F₄ | −28 | −2.4 | 114.05 |
| C₄H₄F₄ | 19 | 20 | 128.08 |
| C₄H₃F₅ | 15 | 36 | 146.07 |
| C₄H₂F₆ | 5.4 | 38 | 164.06 |
| C₅H₄F₆ | 47 | 49 | 178.09 |
| C₄H₁F₇ | 18 | 18 | 182.05 |
| C₅H₃F₇ | 32 | 58 | 196.08 |
| C₄F₈ | 0.4 | 6.5 | 200.04 |
| C₅H₂F₈ | 51 | 51 | 214.07 |
| C₅H₁F₉ | 32 | 33 | 232.06 |
| C₆H₃F₉ | 34 | 75 | 246.09 |
| C₅F₁₀ | 26 | 30 | 250.05 |

Hydrofluoric acid (HF) splitting can be carried out with aluminum fluoride (AlF₃) in particular having high surface area.

Suitable catalysts and procedures are described in International Patent Application WO 2009/010472 (application number PCT/EP2008/059112) the contents of which are incorporated by reference into the present patent application. The catalyst described therein is a high surface metal fluoride catalyst which may be supported on a carrier. Aluminium fluoride is the preferred high surface catalyst. The synthesis of such catalysts is described in US patent application publication 2006/0052649 and EP-A-1 666 411. A metal alcoxide is reacted with a fluorinating agent to form the amorphous metal fluoride which is activated by treatment with hydrofluorocarbons or hydrochlorofluorocarbons.

The dehydrofluorination is preferably performed at a temperature from 50 to 500° C., preferably from 250 to 400° C.

Alternatively, the dehydrofluorination can be performed with conventional dehydrofluorination catalysts, e.g. AlF₃, or by applying a base, for example, NaOH or KOH.

The hydrofluoroolefins obtainable according to the process according to the invention are useful as foam blowing agent, in particular for polyurethane or polyisocyanurate foams. They are more particularly useful for manufacture of rigid polyurethane foams, for example as insulating materials.

Said hydrofluoroolefins are also useful as blowing agent for thermoplastic foams, in particular polyalkenyl foams more particularly extruded polystyrene foams.

Preferred compounds for this purpose are those with 6 or less carbon atoms, especially 5 or less carbon atoms. Hydrofluoroolefins having isomers with a boiling point in the range of 0 to 60° C., especially 25 to 50° C. are highly suitable.

Most preferably, (E)-CF₃—CH═CF—CH₂—CF₃ and (Z)—CF₃—CH═CF—CH₂—CF₃ and mixtures thereof are applied as blowing agent. The hydrofluoroolefin can be applied together with other compounds and additives. For example, they can be applied together with one or more other blowing agents, e.g. with alkanes, e.g. with propane, n-butane, iso-butane, pentane, cyclopropane, cyclobutane, cyclopentane, alkenes, hydrofluoroalkanes, e.g. difluoromethane, tetrafluoroethane, pentafluoropropane, hexafluoropropane, heptafluoropropane, hydrofluoroalkenes, e.g. those with 2 to 5 carbon atoms, alcohols, e.g. methanol, or carbon dioxide.

The hydrofluoroolefins can be applied as a premix with polyester polyols or polyether polyols and optionally flame retardants, e.g. phosphate esters or phosphonate esters, as described in WO 02/092676. These premixes are reacted with isocyanates and form polyurethane foams.

The hydrofluoroolefins obtainable according to the invention may also be used as solvent, more particularly as component in solvent mixtures. For example, they can be applied together with at least one solvent selected from the group of linear or branched C₃ to C₈ alkanes, alcohols, chlorinated alkenes and chlorinated alkanes. If the solvent mixture contains one or more alkanes, the content of the alkane or alkanes is preferably in the range of 5% by weight to 95% by weight. If the solvent mixture contains an alcohol, the content of the alcohol is preferably in the range of 1 to 20% by weight.

If a chlorinated alkene or chlorinated alkane is contained in the solvent mixture, the content of the chlorinated alkene or chlorinated alkane is preferably in the range of 5 to 95% by weight of the solvent mixture. A preferred alkene is selected from the group consisting of 1,2-dichloroethylenes. Most preferably, the chlorinated alkene is 1,2-trans-dichloroethylene. The content of 1,2-trans-dichloroethylene is preferably from 5 to 60% by weight of the solvent mixture.

The solvent mixture may also contain a stabilizer, e.g. a stabilizer which protects the components against oxidation or polymerization. It is assumed that polymerization may especially be caused by Lewis acids and Lewis bases. Suitable stabilizers are, for example, epoxides, alkenes, nitroalkanes, diketones, alcohols, bromoalkanes and bromoalcohols. Such stabilizers are disclosed in WO 2008/095881 on page 6. Non-limiting examples are 1,2-epoxypropane, epichlorohydrine, butenes, nitromethane, acetyl acetone, 1,4-benzochinone, methanol, ethanol and isopropanol. If present as a stabilizer, these compounds are contained in an amount of 0.1 to 1% by weight in the total solvent mixture. Other suitable stabilizers are described in U.S. Pat. No. 7,253,327. The stabilizers described therein stabilize hydrofluoroalkanes against dehydrofluorination caused by Lewis acids, e.g. iron halides. The stabilizers are selected from the group of alcohols, amines, amides, nitriles and phosphorous-containing compounds. Diols, e.g. ethylene glycol, alkanolamines, alkylamines, e.g. ethanolamine, n-butylamine, n-propyl amine, diethyl amine and triethyl amine, acetonitrile, adiponitrile, N,N-dimethylformaide, N-methylpyrrolidone, trialkylphosphin oxides and trialkyl phosphates are very suitable. These are preferably of formulae (R¹R²R³)PO and (R¹O)(R²O)(R³O)PO. R¹, R² and R³ are the same or different and denote preferably a C₃ to C₁₀ alkyl group. The alkyl groups are preferably selected from n-butyl, n-hexyl and n-octyl.

The hydrofluoroalkenes are also useful as intermediates in chemical synthesis. For example, in a specific embodiment of the invention, the chlorinated precursor is provided by the combination of a step wherein the chlorinated alkene is reacted with a chlorine-containing compound, such as Cl₂, CCl₄, CCl₃—CCl₃ (e.g., reactant 2 in Table 1), followed by chlorination of the resulting chlorinated alkane (e.g., the chlorinated alkane compound identified as ‘reactant 1’ in table 1)

For example, CH₂═CCl—CH₂—CF₃ is reacted according to reaction 8 of table 1 with CCl₄ in the presence of tert-butylamine and CuCl₂ to form CCl₃—CH₂—CCl₂—CH₂—CF₃. This intermediate is then photochemically chlorinated with chlorine to form CCl₃—CHCl—CCl₂—CH₂—CF₃, CCl₃—CCl₂—CCl₂—CH₂—CF₃, CCl₃—CHCl—CCl₂—CHCl—CF₃, CCl₃—CCl₂—CCl₂—CHCl—CF₃, CCl₃—CCl₂—CCl₂—CCl₂—CF₃ and CCl₃—CHCl—CCl₂—CCl₂—CF₃. The resulting chlorinated precursor is then fluorinated to form CF₃—CHF—CF₂—CH₂—CF₃, CF₃—CF₂—CF₂—CH₂—CF₃, CF₃—CHF—CF₂—CHF—CF₃, CF₃—CF₂—CF₂—CHF—CF₃, CF₃—CF₂—CF₂—CF₂—CF₃ and CF₃—CHF—CF₂—CF₂—CF₃, These fluorinated alkanes are then dehydrofluorinated in step c) to form the respective hydrofluoroalkene.

Some of the compounds of tables 1, 2, and 3a to 3i are assumed to be known.

The compounds considered known are (E)-1,3,3,3-tetrafluoro-propene, (Z)-1,3,3,3-tetrafluoro-propene, (E)-1,1,1,2,3,4,4,4-octafluoro-but-2-ene, (Z)-1,1,1,2,3,4,4,4-octafluoro-but-2-ene, 1,1,2,3,3,4,4,4-octafluoro-but-1-ene, (E)-1,1,1,2,3,4,4,4-octafluoro-but-2-ene, (Z)-1,1,1,2,4,4,4-heptafluoro-but-2-ene, (Z)-1,1,1,2,4,4,4-heptafluoro-but-2-ene, (E)-1,2,3,3,4,4,4-heptafluoro-but-1-ene, (Z)-1,2,3,3,4,4,4-heptafluoro-but-1-ene, 1,1,2,3,4,4,4-heptafluoro-but-1-ene, (E)-1,1,1,2,3,4,4-heptafluoro-but-2-ene, (Z)-1,1,1,2,3,4,4-heptafluoro-but-2-ene, (E)-1,1,1,2,3,4,4-heptafluoro-but-2-ene, (Z)-1,1,1,2,3,4,4-heptafluoro-but-2-ene, (E)-1,2,3,3,4,4,4-heptafluoro-but-1-ene, (Z)-1,2,3,3,4,4,4-heptafluoro-but-1-ene, (E)-1,3,3,4,4,4-hexafluoro-but-1-ene, (Z)-1,3,3,4,4,4-hexafluoro-but-1-ene, (E)-1,2,3,4,4,4-hexafluoro-but-1-ene, (E)-1,2,3,4,4,4-hexafluoro-but-1-ene, 2,3,3,4,4,4-hexafluoro-but-1-ene, (E)-1,1,1,2,3-Pentafluoro-but-2-ene, (Z)-1,1,1,2,3-Pentafluoro-but-2-ene, (E)-1,1,1,2,3,4,4,5,5,5-decafluoro-pent-2-ene, (Z)-1,1,1,2,3,4,4,5,5,5-decafluoro-pent-2-ene, (E)-1,1,1,2,3,4,4,5,5,5-decafluoro-pent-2-ene, (Z)-1,1,1,2,3,4,4,5,5,5-decafluoro-pent-2-ene, (E)-1,1,1,2,3,4,4,5,5,5-decafluoro-pent-2-ene, (Z)-1,1,1,2,3,4,4,5,5,5-decafluoro-pent-2-ene, (Z)-1,1,1,2,4,4,5,5,5-nonafluoro-pent-2-ene, (E)-1,1,1,3,4,4,5,5,5-nonafluoro-pent-2-ene, (Z)-1,1,1,3,4,4,5,5,5-nonafluoro-pent-2-ene, 1,1,2,3,3,4,4,4-Octafluoro-but-1-ene and 1,1,3,3,3-Pentafluoro-2-trifluoromethyl-propene. Of these, preferred compounds are those having at least one hydrogen atom and equal to or more than 6 fluorine atoms.

The invention also concerns novel hydrofluoroolefins and novel hydrofluoroolefin isomers identified in the appended Table 1, 2, and 3a-3i.

TABLE 1

| # | Reaction | Reactant1 | Reactant2 | Intermediate |
|---|---|---|---|---|
| 1 | 1 | CH2═CCl—CH3 | CCl3—CCl3 | CCl3—CCl2—CH2—CCl2—CH3 |
| 2 | 2 | CH2═CHCl | CCl3—CCl3 | CCl3—CCl2—CH2—CHCl2 |
| 3 | 3 | CH2═CCl—CCl3 | CCl3—CCl3 | CCl3—CCl2—CH2—CCl2—CCl3 |
| 4 | 4 | CH2═CCl—CH2—CF3 | CCl3—CCl3 | CCl3—CCl2—CH2—CCl2—CH2—CF3 |
| 5 | | | | |
| 6 | 5 | CH2═CCl—CH3 | CCl4 | CCl3—CH2—CCl2—CH3 |
| 7 | 6 | CH2═CHCl | CCl4 | CCl3—CH2—CHCl2 |
| 8 | 7 | CH2═CCl—CCl3 | CCl4 | CCl3—CH2—CCl2—CCl3 |
| 9 | 8 | CH2═CCl—CH2—CF3 | CCl4 | CCl3—CH2—CCl2—CH2—CF3 |
| 10 | | | | |
| 11 | 9 | CH2═CCl—CH2—CF3 | Cl2 | CH2Cl—CCl2—CH2—CF3 |
| 12 | 10 | CH2═CCl—CH2—CF3 | 2Cl2 | CHCl2—CCl2—CH2—CF3 |
| 13 | 11 | CH2═CCl—CH2—CF3 | 2Cl2 | CH2Cl—CCl2—CHCl—CF3 |
| 14 | 12 | CH2═CCl—CH2—CF3 | 3Cl2 | CCl3—CCl2—CH2—CF3 |
| 15 | 13 | CH2═CCl—CH2—CF3 | 3Cl2 | CH2Cl—CCl2—CCl2—CF3 |
| 16 | 14 | CH2═CCl—CH2—CF3 | 3Cl2 | CHCl2—CCl2—CHCl—CF3 |
| 17 | 15 | CH2═CCl—CH2—CF3 | 4Cl2 | CCl3—CCl2—CHCl—CF3 |
| 18 | 16 | CH2═CCl—CH2—CF3 | 4Cl2 | CHCl2—CCl2—CCl2—CF3 |
| 19 | | | | |
| 20 | 17 | CH3—CCl═CH—CF3 | Cl2 | CH3—CCl2—CHCl—CF3 |
| 21 | 18 | CH3—CCl═CH—CF3 | 2Cl2 | CH2Cl—CCl2—CHCl—CF3 |
| 22 | 19 | CH3—CCl═CH—CF3 | 2Cl2 | CH3—CCl2—CCl2—CF3 |
| 23 | 20 | CH3—CCl═CH—CF3 | 3Cl2 | CHCl2—CCl2—CHCl—CF3 |
| 24 | 21 | CH3—CCl═CH—CF3 | 3Cl2 | CH2Cl—CCTl2—CCl2—CF3 |
| 25 | 22 | CH3—CCl═CH—CF3 | 4Cl2 | CCl3—CCl2—CHCl—CF3 |
| 26 | 23 | CH3—CCl═CH—CF3 | 4Cl2 | CHCl2—CCl2—CCl2—CF3 |
| 27 | | | | |
| 28 | 24 | CCl3—CH2—CCl2—CH2—CF3 | Cl2 | CCl3—CHCl—CCl2—CH2—CF3 |
| 29 | 25 | CCl3—CH2—CCl2—CH2—CF3 | 2Cl2 | CCl3—CCl2—CCl2—CH2—CF3 |
| 30 | 26 | CCl3—CH2—CCl2—CH2—CF3 | 2Cl2 | CCl3—CHCl—CCl2—CHCl—CF3 |
| 31 | 27 | CCl3—CH2—CCl2—CH2—CF3 | 3Cl2 | CCl3—CCl2—CCl2—CHCl—CF3 |
| 32 | 28 | CCl3—CH2—CCl2—CH2—CF3 | 3Cl2 | CCl3—CHCl—CCl2—CCl2—CF3 |
| 33 | 29 | CCl3—CH2—CCl2—CH2—CF3 | 3Cl2 | CCl3—CCl2—CCl2—CHCl—CF3 |

| # | Fluorinated Alkane | X | Olefine | Code | C | H | F | F/H | Isomeres |
|---|---|---|---|---|---|---|---|---|---|
| 1 | CF3—CF2—CH2—CF2—CH3 | 7 | 1456 | 540 | 5 | 4 | 6 | 1.5 | 5 |
| 2 | CF3—CF2—CH2—CHF2 | 7 | 1336 | 420 | 4 | 2 | 6 | 3.0 | 4 |
| 3 | CF3—CF2—CH2—CF2—CF3 | 10 | 1429 | 510 | 5 | 1 | 9 | 9.0 | 4 |
| 4 | CF3—CF2—CH2—CF2—CH2—CF3 | 10 | 1549 | 630 | 6 | 3 | 9 | 3.0 | 6 |
| 5 | | | | | | | | | |
| 6 | CF3—CH2—CF2—CH3 | 5 | 1354 | 440 | 4 | 4 | 4 | 1.0 | 3 |
| 7 | CF3—CH2—CHF2 | 5 | 1234 | 320 | 3 | 2 | 4 | 2.0 | 2 |
| 8 | CF3—CH2—CF2—CF3 | 8 | 1327 | 410 | 4 | 1 | 7 | 7.0 | 2 |
| 9 | CF3—CH2—CF2—CH2—CF3 | 8 | 1447 | 530 | 5 | 3 | 7 | 2.3 | 2 |
| 10 | | | | | | | | | |
| 11 | CH2F—CF2—CH2—CF3 | 6 | 1345 | 430 | 4 | 3 | 5 | 1.7 | 4 |
| 12 | CHF2—CF2—CH2—CF3 | 7 | 1336 | 420 | 4 | 2 | 6 | 3.0 | 4 |
| 13 | CH2F—CF2—CHF—CF3 | 7 | 1336 | 420 | 4 | 2 | 6 | 3.0 | 4 |
| 14 | CF3—CF2—CH2—CF3 | 8 | 1327 | 410 | 4 | 1 | 7 | 7.0 | 2 |
| 15 | CH2F—CF2—CF2—CF3 | 8 | 1327 | 410 | 4 | 1 | 7 | 7.0 | 2 |
| 16 | CHF2—CF2—CHF—CF3 | 8 | 1327 | 410 | 4 | 1 | 7 | 7.0 | 3 |
| 17 | CF3—CF2—CHF—CF3 | 9 | 1318 | 400 | 4 | 0 | 8 | per | 2 |
| 18 | CHF2—CF2—CF2—CF3 | 9 | 1318 | 400 | 4 | 0 | 8 | per | 1 |
| 19 | | | | | | | | | |
| 20 | CH3—CF2—CHF—CF3 | 6 | 1345 | 430 | 4 | 3 | 5 | 1.7 | 3 |
| 21 | CH2F—CF2—CHF—CF3 | 7 | 1336 | 420 | 4 | 2 | 6 | 3.0 | 4 |
| 22 | CH3—CF2—CF2—CF3 | 7 | 1336 | 420 | 4 | 2 | 6 | 3.0 | 1 |
| 23 | CHF2—CF2—CHF—CF3 | 8 | 1327 | 410 | 4 | 1 | 7 | 7.0 | 3 |
| 24 | CH2F—CF2—CF2—CF3 | 8 | 1327 | 410 | 4 | 1 | 7 | 7.0 | 2 |
| 25 | CF3—CF2—CHF—CF3 | 9 | 1318 | 400 | 4 | 0 | 8 | per | 2 |
| 26 | CHF2—CF2—CF2—CF3 | 9 | 1318 | 400 | 4 | 0 | 8 | per | 1 |
| 27 | | | | | | | | | |
| 28 | CF3—CHF—CF2—CH2—CF3 | 9 | 1438 | 520 | 5 | 2 | 8 | 4.0 | 4 |
| 29 | CF3—CF2—CF2—CH2—CF3 | 10 | 1429 | 510 | 5 | 1 | 9 | 9.0 | 2 |
| 30 | CF3—CHF—CF2—CHF—CF3 | 10 | 1429 | 510 | 5 | 1 | 9 | 9.0 | 2 |
| 31 | CF3—CF2—CF2—CHF—CF3 | 11 | 141-10 | 500 | 5 | 0 | 10 | per | 2 |
| 32 | CF3—CHF—CF2—CF2—CF3 | 11 | 141-10 | 500 | 5 | 0 | 10 | per | 2 |
| 33 | CF3—CF2—CF2—CHF—CF3 | 11 | 141-10 | 500 | 5 | 0 | 10 | per | 2 |

| # | MF | MW | Alkene isomere1 - E | Alkene isomere2 - Z |
|---|---|---|---|---|
| 1 | C5H4F6 | 178 | CF3—CF═CH—CF2—CH3 | CF3—CF═CH—CF2—CH3 |
| 2 | C4H2F6 | 164 | CF3—CF═CH—CHF2 | CF3—CF═CH—CHF2 |
| 3 | C5H1F9 | 232 | CF3—CF═CH—CF2—CF3 | CF3—CF═CH—CF2—CF3 |
| 4 | C6H3F9 | 246 | CF3—CF═CH—CF2—CH2—CF3 | CF3—CF═CH—CF2—CH2—CF3 |
| 5 | | | | |
| 6 | C4H4F4 | 128 | CF3—CH═CF—CH3 | CF3—CH═CF—CH3 |
| 7 | C3H2F4 | 114 | CF3—CH═CHF | CF3—CH═CHF |
| 8 | C4H1F7 | 182 | CF3—CH═CF—CF3 | CF3—CH═CF—CF3 |
| 9 | C5H3F7 | 196 | CF3—CH═CF—CH2—CF3 | CF3—CH═CF—CH2—CF3 |
| 10 | | | | |
| 11 | C4H3F5 | 146 | CHF═CF—CH2—CF3 | CHF═CF—CH2—CF3 |
| 12 | C4H2F6 | 164 | CF2═CF—CH2—CF3 | CF2═CF—CH2—CF3 |
| 13 | C4H2F6 | 164 | CHF═CF—CHF—CF3 | CHF═CF—CHF—CF3 |
| 14 | C4H1F7 | 182 | CF3—CF═CH—CF3 | CF3—CF═CH—CF3 |
| 15 | C4H1F7 | 182 | CHF═CF—CF2—CF3 | CHF═CF—CF2—CF3 |
| 16 | C4H1F7 | 182 | CF2═CF—CHF—CF3 | |
| 17 | C4F8 | 200 | CF3—CF═CF—CF3 | CF3—CF═CF—CF3 |
| 18 | C4F8 | 200 | CF2═CF—CF2—CF3 | |
| 19 | | | | |
| 20 | C4H3F5 | 146 | CH2═CF—CHF—CF3 | |
| 21 | C4H2F6 | 164 | CHF═CF—CHF—CF3 | CHF═CF—CHF—CF3 |
| 22 | C4H2F6 | 164 | CH2═CF—CF2—CF3 | |
| 23 | C4H1F7 | 182 | CF2—CF—CHF—CF3 | |
| 24 | C4H1F7 | 182 | CHF═CF—CF2—CF3 | CHF═CF—CF2—CF3 |
| 25 | C4F8 | 200 | CF3—CF═CF—CF3 | CF3—CF═CF—CF3 |
| 26 | C4F8 | 200 | CF2═CF—CF2—CF3 | |
| 27 | | | | |
| 28 | C5H2F8 | 214 | CF3—CF═CF—CH2—CF3 | CF3—CF═CF—CH2—CF3 |
| 29 | C5H1F9 | 232 | CF3—CF2—CF═CH—CF3 | CF3—CF2—CF═CH—CF3 |
| 30 | C5H1F9 | 232 | CF3—CF═CF—CHF—CF3 | CF3—CF═CF—CHF—CF3 |
| 31 | C5F10 | 250 | CF3—CF2—CF═CF—CF3 | CF3—CF2—CF═CF—CF3 |
| 32 | C5F10 | 250 | CF3—CF2—CF═CF—CF3 | CF3—CF2—CF═CF—CF3 |
| 33 | C5F10 | 250 | CF3—CF2—CF═CF—CF3 | CF3—CF2—CF═CF—CF3 |

| # | Alkene isomere3 - E | Alkene isomere4 - Z |
|---|---|---|
| 1 | CF3—CF2—CH═CF—CH3 | CF3—CF2—CH═CF—CH3 |
| 2 | CF3—CF2—CH═CHF | CF3—CF2—CH═CHF |
| 3 | | |
| 4 | CF3—CF2—CH═CF—CH2—CF3 | CF3—CF2—CH═CF—CH2—CF3 |
| 5 | | |
| 6 | CF3—CH2—CF═CH2 | |
| 7 | | |
| 8 | | |
| 9 | | |
| 10 | | |
| 11 | CH2F—CF═CH—CF3 | CH2F—CF═CH—CF3 |
| 12 | CHF2—CF═CH—CF3 | CHF2—CF═CH—CF3 |
| 13 | CH2F—CF═CF—CF3 | CH2F—CF═CF—CF3 |
| 14 | | |
| 15 | | |
| 16 | CHF2—CF═CF—CF3 | CHF2—CF═CF—CF3 |
| 17 | | |
| 18 | | |
| 19 | | |
| 20 | CH3—CF═CF—CF3 | CH3—CF═CF—CF3 |
| 21 | CH2F—CF═CF—CF3 | CH2F—CF═CF—CF3 |
| 22 | | |
| 23 | CHF2—CF═CF—CF3 | CHF2—CF═CF—CF3 |
| 24 | | |
| 25 | | |
| 26 | | |
| 27 | | |
| 28 | CF3—CHF—CF═CH—CF3 | CF3—CHF—CF═CH—CF3 |
| 29 | | |
| 30 | | |
| 31 | | |
| 32 | | |
| 33 | | |

| # | Alkene isomere5 - E | Alkene isomere6 - Z |
|---|---|---|
| 1 | CF3—CF2—CH2—CF═CH2 | |
| 2 | | |
| 3 | | |
| 4 | CF3—CF2—CH2—CF═CH—CF3 | CF3—CF2—CH2—CF═CH—CF3 |
| 5 | | |
| 6 | | |
| 7 | | |
| 8 | | |
| 9 | | |
| 10 | | |
| 11 | | |
| 12 | | |
| 13 | | |
| 14 | | |
| 15 | | |
| 16 | | |
| 17 | | |
| 18 | | |
| 19 | | |
| 20 | | |
| 21 | | |
| 22 | | |
| 23 | | |
| 24 | | |
| 25 | | |
| 26 | | |
| 27 | | |
| 28 | | |
| 29 | | |
| 30 | | |
| 31 | | |
| 32 | | |
| 33 | | |

TABLE 2

| # | Reaction | Olefine | MF | MW | Alkene isomere1 - E | Alkene isomere2 - Z |
|---|---|---|---|---|---|---|
| 7 | 6 | 1234 | C3H2F4 | 114 | CF3—CH═CHF | CF3—CH═CHF |
| 17 | 15 | 1318 | C4F8 | 200 | CF3—CF═CF—CF3 | CF3—CF═CF—CF3 |
| 18 | 16 | 1318 | C4F8 | 200 | CF2═CF—CF2—CF3 | |
| 25 | 22 | 1318 | C4F8 | 200 | CF3—CF═CF—CF3 | CF3—CF═CF—CF3 |
| 26 | 23 | 1318 | C4F8 | 200 | CF2═CF—CF2—CF3 | |
| 8 | 7 | 1327 | C4H1F7 | 182 | CF3—CH═CF—CF3 | CF3—CH═CF—CF3 |
| 14 | 12 | 1327 | C4H1F7 | 182 | CF3—CF═CH—CF3 | CF3—CF═CH—CF3 |
| 15 | 13 | 1327 | C4H1F7 | 182 | CHF═CF—CF2—CF3 | CHF═CF—CF2—CF3 |
| 16 | 14 | 1327 | C4H1F7 | 182 | CF2═CF—CF2—CF3 | |
| 23 | 20 | 1327 | C4H1F7 | 182 | CF2═CF—CF2—CF3 | |
| 24 | 21 | 1327 | C4H1F7 | 182 | CHF═CF—CF2—CF3 | CHF═CF—CF2—CF3 |
| 2 | 2 | 1336 | C4H2F6 | 164 | CF3—CF═CH—CHF2 | CF3—CF═CH—CHF2 |
| 12 | 10 | 1336 | C4H2F6 | 164 | CF2═CF—CH2—CF3 | |
| 13 | 11 | 1336 | C4H2F6 | 164 | CHF═CF—CHF—CF3 | CHF═CF—CHF—CF3 |
| 21 | 18 | 1336 | C4H2F6 | 164 | CHF═CF—CHF—CF3 | CHF═CF—CHF—CF3 |
| 22 | 19 | 1335 | C4H2F6 | 164 | CH2═CF—CF2—CF3 | |
| 11 | 9 | 1345 | C4H3F5 | 146 | CHF═CF—CH2—CF3 | CHF═CF—CH2—CF3 |
| 20 | 17 | 1345 | C4H3F5 | 146 | CH2═CF—CHF—CF3 | |
| 6 | 5 | 1354 | C4H4F4 | 128 | CF3—CH═CF—CH3 | CF3—CH═CF—CH3 |
| 31 | 27 | 141-10 | C5F10 | 250 | CF3—CF2—CF═CF—CF3 | CF3—CF2—CF═CF—CF3 |
| 32 | 28 | 141-10 | C5F10 | 250 | CF3—CF2—CF═CF—CF3 | CF3—CF2—CF═CF—CF3 |
| 33 | 29 | 141-10 | C5F10 | 250 | CF3—CF2—CF═CF—CF3 | CF3—CF2—CF═CF—CF3 |
| 3 | 3 | 1429 | C5H1F9 | 232 | CF3—CF═CH—CF2—CF3 | CF3—CF═CH—CF2—CF3 |
| 29 | 25 | 1429 | C5H1F9 | 232 | CF3—CF2—CF═CH—CF3 | CF3—CF2—CF═CH—CF3 |
| 30 | 26 | 1429 | C5H1F9 | 232 | CF3—CF═CF—CHF—CF3 | CF3—CF═CF—CHF—CF3 |
| 28 | 24 | 1438 | C5H2F8 | 214 | CF3—CF═CF—CH2—CF3 | CF3—CF═CF—CH2—CF3 |
| 9 | 8 | 1447 | C5H3F7 | 196 | CF3—CH═CF—CH2—CF3 | CF3—CH═CF—CH2—CF3 |
| 1 | 1 | 1456 | C5H4F6 | 178 | CF3—CF═CH—CF2—CH3 | CF3—CF═CH—CF2—CH3 |
| 4 | 4 | 1549 | C6H3F9 | 246 | CF3—CF═CH—CF2—CH2—CF3 | CF3—CF═CH—CF2—CH2—CF3 |

| # | Alkene isomere3 - E | Alkene isomere4 - Z |
|---|---|---|
| 7 | | |
| 17 | | |
| 18 | | |
| 25 | | |
| 26 | | |
| 8 | | |
| 14 | | |
| 15 | | |
| 16 | CHF2—CF═CF—CF3 | CHF2—CF═CF—CF3 |
| 23 | CHF2—CF═CF—CF3 | CHF2—CF═CF—CF3 |
| 24 | | |
| 2 | CF3—CF2—CH═CHF | CF3—CF2—CH═CHF |
| 12 | CHF2—CF═CH—CF3 | CHF2—CF═CH—CF3 |
| 13 | CH2F—CF═CF—CF3 | CH2F—CF═CF—CF3 |
| 21 | CH2F—CF═CF—CF3 | CH2F—CF═CF—CF3 |
| 22 | | |
| 11 | CH2F—CF═CH—CF3 | CH2F—CF═CH—CF3 |
| 20 | CH3—CF═CF—CF3 | CH3—CF═CF—CF3 |
| 6 | CF3—CH2—CF═CH2 | |
| 31 | | |
| 32 | | |
| 33 | | |
| 3 | CF3—CF2—CH═CF—CF3 | CF3—CF2—CH═CF—CF3 |
| 29 | | |
| 30 | | |
| 28 | CF3—CHF—CF═CH—CF3 | CF3—CHF—CF═CH—CF3 |
| 9 | | |
| 1 | CF3—CF2—CH═CF—CH3 | CF3—CF2—CH═CF—CH3 |
| 4 | CF3—CF2—CH═CF—CH2—CF3 | CF3—CF2—CH═CF—CH2—CF3 |

| # | Alkene isomere5 - E | Alkene isomere6 - Z |
|---|---|---|
| 7 | | |
| 17 | | |
| 18 | | |
| 25 | | |
| 26 | | |
| 8 | | |
| 14 | | |
| 15 | | |
| 16 | | |
| 23 | | |
| 24 | | |
| 2 | | |
| 12 | | |
| 13 | | |
| 21 | | |
| 22 | | |
| 11 | | |
| 20 | | |
| 6 | | |
| 31 | | |
| 32 | | |
| 33 | | |
| 3 | | |
| 29 | | |
| 30 | | |
| 28 | | |
| 9 | | |
| 1 | CF3—CF2—CH2—CF═CH2 | |
| 4 | CF3—CF2—CH2—CF═CH—CF3 | CF3—CF2—CH2—CF═CH—CF3 |

TABLE 3a

| # | 7 |
|---|---|
| Reaction | 6 |
| Olefine | 1234 |
| MF | C3H2F4 |
| MW | 114 |
| Alkene isomere1 - E | CF3—CH═CHF |
| | (E)-1,3,3,3-tetrafluoro-propene |
| Alkene isomere2 - Z | CF3—CH═CHF |
| | (Z)-1,3,3,3-tetrafluoro-propene |
| Alkene isomere3 - E | |
| Alkene isomere4 - Z | |
| Alkene isomere5 - E | |
| Alkene isomere6 - Z | |
| Beilstein Hits of MF | 12 |
| | (E)-1,3,3,3-Tetrafluoro-propene |
| | (E)-1,2,3,3-Tetrafluoro-propene |
| | 2,3,3,3-Tetrafluoro-propene |
| | −28 |
| | 1,1,3,3-Tetrafluoro-propene |
| | −2.4 |
| | (E)-1,3,3,3-Tetrafluoro-propene |
| | (Z)-1,3,3,3-Tetrafluoro-propene |
| | (Z)-1,2,3,3-Tetrafluoro-propene |

TABLE 3b

| # | 17 | 18 |
|---|---|---|
| Reaction | 15 | 16 |
| Olefine | 1318 | 1318 |
| MF | C4F8 | C4F8 |
| MW | 200 | 200 |
| Alkene isomere1 - E | CF3—CF═CF—CF3 | CF2═CF—CF2—CF3 |
| | (E)-1,1,1,2,3,4,4,4-octafluoro-but-2-ene | 1,1,2,3,3,4,4,4-octafluoro-but-1-ene |
| Alkene isomere2 - Z | CF3—CF═CF—CF3 | |
| | (Z)-1,1,1,2,3,4,4,4-octafluoro-but-2-ene | |
| Alkene isomere3 - E | | |
| Alkene isomere4 - Z | | |
| Alkene isomere5 - E | | |
| Alkene isomere6 - Z | | |
| Beilstein Hits of MF | 15 | |
| nBp (° C.) | (Z)-1,1,1,2,3,4,4,4-Octafluoro-but-2-ene | |
| | 0.4-3 | |
| nBp (° C.) | 1,1,2,3,3,4,4,4-Octafluoro-but-1-ene | |
| | 4.8 | |
| nBp (° C.) | 1,1,3,3,3-Pentafluoro-2-trifluoromethyl-propene | |
| | 6.5 | |
| nBp (° C.) | (E)-1,1,1,2,3,4,4,4-Octafluoro-but-2-ene | |
| | 1.2 | |
| # | 25 | 26 |
| Reaction | 22 | 23 |
| Olefine | 1318 | 1318 |
| MF | C4F8 | C4F8 |
| MW | 200 | 200 |
| Alkene isomere1 - E | CF3—CF═CF—CF3 | CF2═CF—CF2—CF3 |
| | (E)-1,1,1,2,3,4,4,4-octafluoro-but-2-ene | 1,1,2,3,3,4,4,4-octafluoro-but-1-ene |
| Alkene isomere2 - Z | CF3—CF═CF—CF3 | |
| | (Z)-1,1,1,2,3,4,4,4-octafluoro-but-2-ene | |
| Alkene isomere3 - E | | |
| Alkene isomere4 - Z | | |
| Alkene isomere5 - E | | |
| Alkene isomere6 - Z | | |

| | |
|---|---|
| Beilstein Hits of MF | 15 |
| nBp (° C.) | (Z)-1,1,1,2,3,4,4,4-Octafluoro-but-2-ene |
| | 0.4-3 |
| nBp (° C.) | 1,1,2,3,3,4,4,4-Octafluoro-but-1-ene |
| | 4.8 |
| nBp (° C.) | 1,1,3,3,3-Pentafluoro-2-trifluoromethyl-propene |
| | 6.5 |
| nBp (° C.) | (E)-1,1,1,2,3,4,4,4-Octafluoro-but-2-ene |
| | 1.2 |

TABLE 3c

| # | 8 | 14 | 15 |
|---|---|---|---|
| Reaction | 7 | 12 | 13 |
| Olefine | 1327 | 1327 | 1327 |
| MF | C4H1F7 | C4H1F7 | C4H1F7 |
| MW | 182 | 182 | 182 |
| Alkene isomere1 - E | CF3—CH═CF—CF3 | CF3—CF═CH—CF3 | CHF═CF—CF2—CF3 |
| | (E)-1,1,1,2,4,4,4-heptafluoro-but-2-ene | (E)-1,1,1,2,4,4,4-heptafluoro-but-2-ene | (E)-1,2,3,3,4,4,4-heptafluoro-but-1-ene |
| Alkene isomere2 - Z | CF3—CH═CF—CF3 | CF3—CF═CH—CF3 | CHF═CF—CF2—CF3 |
| | (Z)-1,1,1,2,4,4,4-heptafluoro-but-2-ene | (Z)-1,1,1,2,4,4,4-heptafluoro-but-2-ene | (Z)-1,2,3,3,4,4,4-heptafluoro-but-1-ene |
| Alkene isomere3 - E | | | |
| Alkene isomere4 - Z | | | |
| Alkene isomere5 - E | | | |
| Alkene isomere6 - Z | | | |

| | |
|---|---|
| Beilstein Hits of MF | 15 |
| nBp (° C.) | 1,3,3,3-Tetrafluoro-2-trifluoromethyl-propene |
| | 17 |
| nBp (° C.) | 1,1,2,3,3,4,4-Heptafluoro-but-1-ene |
| | 20-27 |
| nBp (° C.) | 1,1,2,3,4,4,4-Heptafluoro-but-1-ene |
| nBp (° C.) | 1,1,2,3,4,4,4-Heptafluoro-but-1-ene |
| | 7-8 |
| nBp (° C.) | (Z)-1,1,1,2,4,4,4-Heptafluoro-but-2-ene |
| | 7-10 |
| nBp (° C.) | 1,1,3,3,4,4-4-Heptafluoro-but-1-ene |
| | 10-11 |
| nBp (° C.) | (E)-1,1,1,2,3,4,4-Heptafluoro-but-2-ene |
| nBp (° C.) | (Z)-1,1,1,2,3,4,4-Heptafluoro-but-2-ene |
| | 18 |
| nBp (° C.) | (Z)-1,2,3,3,4,4,4-Heptafluoro-but-1-ene |
| | 18 |
| nBp (° C.) | (E)-1,2,3,3,4,4,4-Heptafluoro-but-1-ene |

| # | 16 | 23 | 24 |
|---|---|---|---|
| Reaction | 14 | 20 | 21 |
| Olefine | 1327 | 1327 | 1327 |
| MF | C4H1F7 | C4H1F7 | C4H1F7 |
| MW | 182 | 182 | 182 |
| Alkene isomere1 - E | CF2═CF—CHF—CF3 | CF2═CF—CHF—CF3 | CHF═CF—CF2—CF3 |
| | 1,1,2,3,4,4,4-heptafluoro-but-1-ene | 1,1,2,3,4,4,4-heptafluoro-but-1-ene | (E)-1,2,3,3,4,4,4-heptafluoro-but-1-ene |
| Alkene isomere2 - Z | | | CHF═CF—CF2—CF3 |
| | | | (Z)-1,2,3,3,4,4,4-heptafluoro-but-1-ene |
| Alkene isomere3 - E | CHF2—CF═CF—CF3 | CHF2—CF═CF—CF3 | |
| | (E)-1,1,1,2,3,4,4-heptafluoro-but-2-ene | (E)-1,1,1,2,3,4,4-heptafluoro-but-2-ene | |
| Alkene isomere4 - Z | CHF2—CF=CF—CF3 | CHF2—CF=CF—CF3 | |
| | (Z)-1,1,1,2,3,4,4-heptafluoro-but-2-ene | (Z)-1,1,1,2,3,4,4-heptafluoro-but-2-ene | |
| AlRene isomere5 - E | | | |
| Alkene isomere6 - Z | | | |

| | |
|---|---|
| Beilstein Hits of MF | 15 |
| nBp (° C.) | 1,3,3,3-Tetrafluoro-2-trifluoromethyl-propene |
| | 17 |
| nBp (° C.) | 1,1,2,3,3,4,4-Heptafluoro-but-1-ene |
| | 20-27 |
| nBp (° C.) | 1,1,2,3,4,4,4-Heptafluoro-but-1-ene |
| nBp (° C.) | 1,1,2,3,4,4,4-Heptafluoro-but-1-ene |
| | 7-8 |
| nBp (° C.) | (Z)-1,1,1,2,4,4,4-Heptafluoro-but-2-ene |
| | 7-10 |
| nBp (° C.) | 1,1,3,3,4,4-4-Heptafluoro-but-1-ene |
| | 10-11 |
| nBp (° C.) | (E)-1,1,1,2,3,4,4-Heptafluoro-but-2-ene |
| nBp (° C.) | (Z)-1,1,1,2,3,4,4-Heptafluoro-but-2-ene |
| | 18 |
| nBp (° C.) | (Z)-1,2,3,3,4,4,4-Heptafluoro-but-1-ene |
| | 18 |
| nBp (° C.) | (E)-1,2,3,3,4,4,4-Heptafluoro-but-1-ene |

TABLE 3d

| # | 2 | 12 | 13 |
|---|---|---|---|
| Reaction | 2 | 10 | 11 |
| Olefine | 1336 | 1336 | 1336 |
| MF | C4H2F6 | C4H2F6 | C4H2F6 |
| MW | 164 | 164 | 164 |
| Alkene isomere1 - E | CF3—CF═CH—CHF2 | CF2═CF—CH2—CF3 | CHF═CF—CHF—CF3 |
| | (E)-1,1,1,2,4,4-hexafluoro-but-2-ene | 1,1,2,4,4-hexafluoro-but-1-ene | (E)-1,2,3,4,4,4-hexafluoro-but-1-ene |
| Alkene isomere2 - Z | CF3—CF═CH—CHF2 | | CHF═CF—CHF—CF3 |
| | (Z)-1,1,1,2,4,4-hexafluoro-but-2-ene | | (Z)-1,2,3,4,4,4-hexafluoro-but-1-ene |
| Alkene isomere3 - E | CF3—CF2—CH═CHF | CHF2—CF═CH—CF3 | CH2F—CF═CF—CF3 |
| | (E)-1,3,3,4,4,4-hexafluoro-but-1-ene | (E)-1,1,1,3,4,4-hexafluoro-but-2-ene | (E)-1,1,1,2,3,4-hexafluoro-but-2-ene |
| Alkene isomere4 - Z | CF3—CF2—CH═CHF | CHF2—CF═CH—CF3 | CH2F—CF═CF—CF3 |
| | (Z)-1,3,3,4,4,4-hexafluoro-but-1-ene | (Z)-1,1,1,3,4,4-hexafluoro-but-2-ene | (Z)-1,1,1,2,3,4-hexafluoro-but-2-ene |
| Alkene isomere5 - E | | | |
| Alkene isomere6 - Z | | | |

| | |
|---|---|
| Beilstein Hits of MF | 27 |
| nBp (° C.) | (Z)-1,1,1,4,4,4-Hexafluoro-but-2-ene |
| | 33-38 |
| nBp (° C.) | (E)-1,1,1,4,4,4-Hexafluoro-but-2-ene |
| | 6 |
| nBp (° C.) | 2,3,3,4,4,4-Hexafluoro-but-1-ene |
| | 3-7 |
| nBp (° C.) | (E)-1,1,1,4,4,4-Hexafluoro-but-2-ene |
| | 5.4-6.4 |
| nBp (° C.) | 3,3,3-Trifluoro-2-trifluoromethyl-propene |
| | 13 |
| nBp (° C.) | (E)-1,1,2,3,4,4-Hexafluoro-but-2-ene |
| nBp (° C.) | (Z)-1,1,2,3,4,4-Hexafluoro-but-2-ene |
| nBp (° C.) | (E)-1,1,2,3,4,4-Hexafluoro-but-2-ene |
| nBp (° C.) | (E)-1,2,3,4,4,4-Hexafluoro-but-1-ene |
| nBp (° C.) | (Z)-1,3,3,4,4,4-Hexafluoro-but-1-ene |
| nBp (° C.) | (E)-1,3,3,4,4,4-Hexafluoro-but-1-ene |
| nBp (° C.) | (Z)-1,2,3,3,4,4-Hexafluoro-but-1-ene |

| # | 21 | 22 |
|---|---|---|
| Reaction | 18 | 19 |
| Olefine | 1336 | 1336 |
| MF | C4H2F6 | C4H2F6 |
| MW | 164 | 164 |
| Alkene isomere1 - E | CHF═CF—CHF—CF3 | CH2═CF—CF2—CF3 |
| | (E)-1,2,3,4,4,4-hexafluoro-but-1-ene | 2,3,3,4,4,4-hexafluoro-but-1-ene |
| Alkene isomere2 - Z | CHF═CF—CHF—CF3 | |
| | (Z)-1,2,3,4,4,4-hexafluoro-but-1-ene | |
| Alkene isomere3 - E | CH2F—CF═CF—CF3 | |
| | (E)-1,1,1,2,3,4-hexafluoro-but-2-ene | |
| Alkene isomere4 - Z | CH2F—CF═CF—CF3 | |
| | (Z)-1,1,1,2,3,4-hexafluoro-but-2-ene | |
| Alkene isomere5 - E | | |
| Alkene isomere6 - Z | | |

| | |
|---|---|
| Beilstein Hits of MF | 27 |
| nBp (° C.) | (Z)-1,1,1,4,4,4-Hexafluoro-but-2-ene |
| | 33-38 |
| nBp (° C.) | (E)-1,1,1,4,4,4-Hexafluoro-but-2-ene |
| | 6 |
| nBp (° C.) | 2,3,3,4,4,4-Hexafluoro-but-1-ene |
| | 3-7 |
| nBp (° C.) | (E)-1,1,1,4,4,4-Hexafluoro-but-2-ene |
| | 5.4-6.4 |
| nBp (° C.) | 3,3,3-Trifluoro-2-trifluoromethyl-propene |
| | 13 |
| nBp (° C.) | (E)-1,1,2,3,4,4-Hexafluoro-but-2-ene |
| nBp (° C.) | (Z)-1,1,2,3,4,4-Hexafluoro-but-2-ene |
| nBp (° C.) | (E)-1,1,2,3,4,4-Hexafluoro-but-2-ene |
| nBp (° C.) | (E)-1,2,3,4,4,4-Hexafluoro-but-1-ene |
| nBp (° C.) | (Z)-1,3,3,4,4,4-Hexafluoro-but-1-ene |
| nBp (° C.) | (E)-1,3,3,4,4,4-Hexafluoro-but-1-ene |
| nBp (° C.) | (Z)-1,2,3,3,4,4-Hexafluoro-but-1-ene |

TABLE 3e

| # | 11 | 20 |
|---|---|---|
| Reaction | 9 | 17 |
| Olefine | 1345 | 1345 |
| MF | C4H3F5 | C4H3F5 |
| MW | 146 | 146 |
| Alkene isomere1 - E | CHF═CF—CH2—CF3 | CH2═CF—CHF—CF3 |
| | (E)-1,2,4,4,4-Pentafluoro-but-1-ene | 2,3,4,4,4-pentafluoro-but-1-ene |
| Alkene isomere2 - Z | CHF═CF—CH2—CF3 | |
| | (Z)-1,2,4,4,4-Pentafluoro-but-1-ene | |
| Alkene isomere3 - E | CH2F—CF═CH—CF3 | CH3—CF═CF—CF3 |
| | (E)-1,1,1,3,4-Pentafluoro-but-2-ene | (E)-1,1,1,2,3-Pentafluoro-but-2-ene |
| Alkene isomere4 - Z | CH2F—CF═CH—CF3 | CH3—CF═CF—CF3 |
| | (Z)-1,1,1,3,4-Pentafluoro-but-2-ene | (Z)-1,1,1,2,3-Pentafluoro-but-2-ene |
| Alkene isomere5 - E | | |
| Alkene isomere6 - Z | | |

| | |
|---|---|
| Beilstein Hits of MF | 13 |
| nBp (° C.) | 1,1,2,3,3-Pentafluoro-but-1-ene |
| | 24-26 |
| nBp (° C.) | 1,1,4,4,4-Pentafluoro-but-1-ene |
| | 18.6 |
| nBp (° C.) | 3,3,4,4,4-Pentafluoro-but-1-ene |
| | 26 |
| nBp (° C.) | 1,1,3,3,3-Pentafluoro-2-methyl-propene |
| | 15 |
| nBp (° C.) | (E)-1,1,2,4,4-Pentafluoro-but-2-ene |
| nBp (° C.) | (E)-1,1,1,2,3-Pentafluoro-but-2-ene |
| nBp (° C.) | 2-Difluoromethyl-3,3,3-trifluoro-propene |
| | 36 |
| nBp (° C.) | (Z)-1,1,1,2,3-Pentafluoro-but-2-ene |
| nBp (° C.) | (E)-1,1,2,3,4-Pentafluoro-but-2-ene |
| nBp (° C.) | (Z)-1,1,2,4,4-Pentafluoro-but-2-ene |
| nBp (° C.) | (Z)-1,2,3,3,4-Pentafluoro-but-1-ene |
| nBp (° C.) | (Z)-1,1,1,2,4-Pentafluoro-but-2-ene |

| # | 6 |
|---|---|
| Reaction | 5 |
| Olefine | 1354 |
| MF | C4H4F4 |
| MW | 128 |
| Alkene isomere1 - E | CF3—CH═CF—CH3 |
| | (E)-1,1,1,3-tetrafluoro-but-2-ene |
| Alkene isomere2 - Z | CF3—CH═CF—CH3 |
| | (Z)-1,1,1,3-tetrafluoro-but-2-ene |
| Alkene isomere3 - E | CF3—CH2—CF═CH2 |
| | 2,4,4,4-tetrafluoro-but-2-ene |
| Alkene isomere4 - Z | |
| Alkene isomere5 - E | |
| Alkene isomere6 - Z | |
| Beilstein Hits of MF | 11 |
| nBp (° C.) | 1,1,3,3-Tetrafluoro-2-methyl-propene |
| | 19-20 |
| nBp (° C.) | 3,3,4,4-Tetrafluoro-but-1-ene |
| | 27 |
| nBp (° C.) | 2-Difluoromethyl-3,3-difluoro-propene |
| | 55 |
| nBp (° C.) | (E)-1,1,1,2-Tetrafluoro-but-2-ene |
| nBp (° C.) | (Z)-1,1,1,2-Tetrafluoro-but-2-ene |
| nBp (° C.) | (Z)-1,3,3,3-Tetrafluoro-2-methyl-propene |
| nBp (° C.) | (E)-1,3,3,3-Tetrafluoro-2-methyl-propene |
| nBp (° C.) | (E)-1,3,3,3-Tetrafluoro-2-methyl-propene |
| nBp (° C.) | 1,1,4,4-tetrafluoro-1-butene |
| | 38-39 |
| nBp (° C.) | |
| nBp (° C.) | |
| nBp (° C.) | |

TABLE 3f

| # | 31 | 32 |
|---|---|---|
| Reaction | 27 | 28 |
| Olefine | 141-10 | 141-10 |
| MF | C5F10 | C5F10 |
| MW | 250 | 250 |
| Alkene isomere1 - E | CF3—CF2—CF═CF—CF3 | CF3—CF2—CF═CF—CF3 |
| | (E)-1,1,1,2,3,4,4,5,5,5-decafluoro-pent-2-ene | (E)-1,1,1,2,3,4,4,5,5,5-decafluoro-pent-2-ene |
| Alkene isomere2 - Z | CF3—CF2—CF═CF—CF3 | CF3—CF2—CF═CF—CF3 |
| | (Z)-1,1,1,2,3,4,4,5,5,5-decafluoro-pent-2-ene | (Z)-1,1,1,2,3,4,4,5,5,5-decafluoro-pent-2-ene |
| Alkene isomere3 - E | | |
| Alkene isomere4 - Z | | |
| Alkene isomere5 - E | | |
| Alkene isomere6 - Z | | |

| | |
|---|---|
| Beilstein Hits of MF | 11 |
| nBp (° C.) | 1,1,2,3,3,4,4,5,5,5-Decafluoro-pent-1-ene |
| | 29 |
| nBp (° C.) | 1,1,2,3,4,4,4-Heptafluoro-3-trifluoromethyl-but-1-ene |
| | 27 |
| nBp (° C.) | (Z)-1,1,1,2,3,4,4,5,5,5-Decafluoro-pent-2-ene |
| | 26-28 |
| nBp (° C.) | 1,1,1,2,4,4,4-Heptafluoro-3-trifluoromethyl-but-2-ene |
| | 28-30 |
| nBp (° C.) | (E)-1,1,1,2,3,4,4,5,5,5-Decafluoro-pent-2-ene |
| | 26-29 |
| nBp (° C.) | (Z)-1,1,1,2,3,4,4,5,5,5-Decafluoro-pent-2-ene |
| nBp (° C.) | 1,1,3,3,4,4,4-Heptafluoro-2-trifluoromethyl-but-1-ene |

| # | 33 |
|---|---|
| Reaction | 29 |
| Olefine | 141-10 |
| MF | C5F10 |
| MW | 250 |
| Alkene isomere1 - E | CF3—CF2—CF═CF—CF3 |
| | (E)-1,1,1,2,3,4,4,5,5,5-decafluoro-pent-2-ene |
| Alkene isomere2 - Z | CF3—CF2—CF═CF—CF3 |
| | (Z)-1,1,1,2,3,4,4,5,5,5-decafluoro-pent-2-ene |
| Alkene isomere3 - E | |
| Alkene isomere4 - Z | |
| Alkene isomere5 - E | |
| Alkene isomere6 - Z | |
| Beilstein Hits of MF | 11 |
| nBp (° C.) | 1,1,2,3,3,4,4,5,5,5-Decafluoro-pent-1-ene |
| | 29 |
| nBp (° C.) | 1,1,2,3,4,4,4-Heptafluoro-3-trifluoromethyl-but-1-ene |
| | 27 |
| nBp (° C.) | (Z)-1,1,1,2,3,4,4,5,5,5-Decafluoro-pent-2-ene |
| | 26-28 |
| nBp (° C.) | 1,1,1,2,4,4,4-Heptafluoro-3-trifluoromethyl-but-2-ene |
| | 28-30 |
| nBp (° C.) | (E)-1,1,1,2,3,4,4,5, 5,5-Decafluoro-pent-2-ene |
| | 26-29 |
| nBp (° C.) | (Z)-1,1,1,2,3,4,4,5,5,5-Decafluoro-pent-2-ene |
| nBp (° C.) | 1,1,3,3,4,4,4-Heptafluoro-2-trifluoromethyl-but-1-ene |

TABLE 3g

| # | 3 | 29 |
|---|---|---|
| Reaction | 3 | 25 |
| Olefine | 1429 | 1429 |
| MF | C5H1F9 | C5H1F9 |
| MW | 232 | 232 |
| Alkene isomere1 - E | CF3—CF═CH—CF2—CF3 | CF3—CF2—CF═CH—CF3 |
| | (E)-1,1,1,2,4,4,5,5,5-nonafluoro-pent-2-ene | (E)-1,1,1,3,4,4,5,5,5-nonafluoro-pent-2-ene |
| Alkene isomere2 - Z | CF3—CF═CH—CF2—CF3 | CF3—CF2—CF═CH—CF3 |
| | (Z)-1,1,1,2,4,4,5,5,5-nonafluoro-pent-2-ene | (Z)-1,1,1,3,4,4,5,5,5-nonafluoro-pent-2-ene |
| Alkene isomere3 - E | | |
| Alkene isomere4 - Z | | |
| Alkene isomere5 - E | | |
| Alkene isomere6 - Z | | |

| | |
|---|---|
| Beiltein Hits of MF | 18 |
| nBp (° C.) | 1,1,3,3,4,4,5,5,5-Nonafluoro-pent-1-ene |
| | 30-37 |
| nBp (° C.) | 1,1,1,4,4,4-Hexafluoro-2-trifluoromethyl-but-2-ene |
| | 32-33 |
| nBp (° C.) | 1,1,3,4,4,4-Hexafluoro-3-trifluoromethyl-but-1-ene |
| | 30 |
| nBp (° C.) | 1,1,2,3,3,4,4,5,5-Nonafluoro-pent-1-ene |
| | 48-49 |
| nBp (° C.) | (Z)-1,1,1,3,4,4,5,5,5-Nonafluoro-pent-2-ene |
| | 32-33 |
| nBp (° C.) | (E)-1,1,1,3,4,4,5,5,5-Nonafluoro-pent-2-ene |
| nBp (° C.) | (Z)-1,2,3,4,4,4-Hexafluoro-3-trifluoromethyl-but-1-ene |
| | 38-38.5 |
| nBp (° C.) | (Z)-1,1,1,2,4,4,5,5,5-Nonafluoro-pent-2-ene |
| | 28 |
| nBp (° C.) | (E)-1,2,3,3,4,4,5,5,5-Nonafluoro-pent-1-ene |
| | 32-34 |
| nBp (° C.) | (Z)-1,1,1,2,4,4,5,5,5-Nonafluoro-pent-2-ene |
| | 24-26 |
| nBp (° C.) | (Z)-1,2,3,3,4,4,5,5,5-Nonafluoro-pent-1-ene |
| nBp (° C.) | (Z)-1,1,1,2,3,4,4,5,5-Nonafluoro-pent-2-ene |
| nBp (° C.) | (E)-1,1,1,2,3,4,4,5,5-Nonafluoro-pent-2-ene |
| nBp (° C.) | (Z)-1,1,2,3,4,4,5,5,5-Nonafluoro-pent-2-ene |
| nBp (° C.) | (E)-1,1,2,3,4,4,5,5,5-Nonafluoro-pent-2-ene |

| # | 30 |
|---|---|
| Reaction | 26 |
| Olefine | 1429 |
| MF | C5H1F9 |
| MW | 232 |
| Alkene isomere1 - E | CF3—CF═CF—CHF—CF3 |
| | (E)-1,1,1,2,3,4,5,5,5-nonafluoro-pent-2-ene |
| Alkene isomere2 - Z | CF3—CF═CF—CHF—CF3 |
| | (Z)-1,1,1,2,3,4,5,5,5-nonafluoro-pent-2-ene |
| Alkene isomore3 - E | |
| Alkene isomere4 - Z | |
| Alkene isomere5 - E | |
| Alkene isomere6 - Z | |
| Beiltein Hits of MF | 18 |
| nBp (° C.) | 1,1,3,3,4,4,5,5,5-Nonafluoro-pent-1-ene |
| | 30-37 |
| nBp (° C.) | 1,1,1,4,4,4-Hexafluoro-2-trifluoromethyl-but-2-ene |
| | 32-33 |
| nBp (° C.) | 1,1,3,4,4,4-Hexafluoro-3-trifluoromethyl-but-1-ene |
| | 30 |
| nBp (° C.) | 1,1,2,3,3,4,4,5,5-Nonafluoro-pent-1-ene |
| | 48-49 |
| nBp (° C.) | (Z)-1,1,1,3,4,4,5,5,5-Nonafluoro-pent-2-ene |
| | 32-33 |
| nBp (° C.) | (E)-1,1,1,3,4,4,5,5,5-Nonafluoro-pent-2-ene |
| nBp (° C.) | (Z)-1,2,3,4,4,4-Hexafluoro-3-trifluoromethyl-but-1-ene |
| | 38-38.5 |
| nBp (° C.) | (Z)-1,1,1,2,4,4,5,5,5-Nonafluoro-pent-2-ene |
| | 28 |
| nBp (° C.) | (E)-1,2,3,3,4,4,5,5,5-Nonafluoro-pent-1-ene |
| | 32-34 |
| nBp (° C.) | (Z)-1,1,1,2,4,4,5,5,5-Nonafluoro-pent-2-ene |
| | 24-26 |
| nBp (° C.) | (Z)-1,2,3,3,4,4,5,5,5-Nonafluoro-pent-1-ene |
| nBp (° C.) | (Z)-1,1,1,2,3,4,4,5,5-Nonafluoro-pent-2-ene |
| nBp (° C.) | (E)-1,1,1,2,3,4,4,5,5-Nonafluoro-pent-2-ene |
| nBp (° C.) | (Z)-1,1,2,3,4,4,5,5,5-Nonafluoro-pent-2-ene |
| nBp (° C.) | (E)-1,1,2,3,4,4,5,5,5-Nonafluoro-pent-2-ene |

TABLE 3h

| # | 28 | 9 |
|---|---|---|
| Reaction | 24 | 8 |
| Olefine | 1438 | 1447 |
| MF | C5H2F8 | C5H3F7 |
| MW | 214 | 196 |
| Alkene isomere1 - E | CF3—CF═CF—CH2—CF3 | CF3—CH═CF—CH2—CF3 |
| | (E)-1,1,1,2,3,5,5,5-octafluoro-pent-2-ene | (E)-1,1,1,3,5,5,5-Heptafluoro-pent-2-ene |
| Alkene isomere2 - Z | CF3—CF═CF—CH2—CF3 | CF3—CH═CF—CH2—CF3 |
| | (Z)-1,1,1,2,3,5,5,5-octafluoro-pent-2-ene | (Z)-1,1,1,3,5,5,5-Heptafluoro-pent-2-ene |
| Alkene isomere3 - E | CF3—CHF—CF═CH—CF3 | |
| | (E)-1,1,1,3,4,5,5,5-octafluoro-pent-2-ene | |
| Alkene isomere4 - Z | CF3—CHF—CF═CH—CF3 | |
| | (Z)-1,1,1,3,4,5,5,5-octafluoro-pent-2-ene | |
| Alkene isomere5 - E | | |
| Alkene isomere6 - Z | | |
| Beilstein Hits of MF | 13 | 18 |
| nBp (° C.) | 1,1,4,4,4-Pentafluoro-2-trifluoromethyl-but-1-ene | 3,3,4,4,5,5,5-Heptafluoro-pent-1-ene |
| | | 32 |
| nBp (° C.) | (Z)-1,3,4,4,4-Pentafluoro-3-trifluoromethyl-but-1-ene | 1,1,1,3-Tetrafluoro-2-trifluoromethyl-but-2-ene |
| | 51 | 58 |
| nBp (° C.) | (E)-1,3,4,4,4-Pentafluoro-3-trifluoromethyl-but-1-ene | 2,3,3,4,4,5,5-Heptafluoro-pent-1-ene |
| | | 56-58 |
| nBp (° C.) | (Z)-1,3,4,4,4-Pentafluoro-3-trifluoromethyl-but-1-ene | 1,1,3,3,5,5,5-Heptafluoro-pent-1-ene |
| | 49 | 58 |
| nBp (° C.) | (Z)-1,3,3,4,4,5,5,5-Octafluoro-pent-1-ene | (E)-1,1,1,2,4,4,4-Heptafluoro-3-methyl-but-2-ene |
| nBp (° C.) | (E)-1,3,3,4,4,5,5,5-Octafluoro-pent-1-ene | (Z)-1,1,1,2,4,4,4-Heptafluoro-3-methyl-but-2-ene |
| | | 38 |
| nBp (° C.) | 3,3,4,4,4-Pentafluoro-2-trifluoromethyl-but-1-ene | 3,4,4,4-Tetrafluoro-3-trifluoromethyl-but-1-ene |
| | 39-40 | |

TABLE 3i

| # | 1 | 4 |
|---|---|---|
| Reaction | 1 | 4 |
| Olefine | 1456 | 1549 |
| MF | C5H4F6 | C6H3F9 |
| MW | 178 | 246 |
| Alkene isomere1 - E | CF3—CF═CH—CF2—CH3 | CF3—CF═CH—CF2—CH2—CF3 |
| | (E)-1,1,1,2,4,4-hexafluoro-but-2-ene | (E)-1,1,1,2,4,4,6,6,6-Nonafluoro-hex-2-ene |
| Alkene isomere2 - Z | CF3—CF═CH—CF2—CH3 | CF3—CF═CH—CF2—CH2—CF3 |
| | (Z)-1,1,1,2,4,4-hexafluoro-but-2-ene | (Z)-1,1,1,2,4,4,6,6,6-Nonafluoro-hex-2-ene |
| Alkene isomere3 - E | CF3—CF2—CH═CF—CH3 | CF3—CF2—CH═CF—CH2—CF3 |
| | (E)-1,1,1,2,2,4-hexafluoro-but-2-ene | (E)-1,1,1,2,2,4,6,6,6-Nonafluoro-hex-3-ene |
| Alkene isomere4 - Z | CF3—CF2—CH═CF—CH3 | CF3—CF2—CH═CF—CH2—CF3 |
| | (Z)-1,1,1,2,2,4-hexafluoro-but-2-ene | (Z)-1,1,1,2,2,4,6,6,6-Nonafluoro-hex-3-ene |
| Alkene isomere5 - E | CF3—CF2—CH2—CF═CH2 | CF3—CF2—CH2—CF═CH—CF3 |
| | 2,4,4,5,5,5-hexafluoro-but-1-ene | (E)-1,1,1,3,5,5,6,6,6-Nonafluoro-hex-2-ene |
| Alkene isomere6 - Z | | CF3—CF2—CH2—CF═CH—CF3 |
| | | (Z)-1,1,1,3,5,5,6,6,6-Nonafluoro-hex-2-ene |
| Beilstein Hits of MF | 16 | 26 |
| nBp (° C.) | (E)-1,1,1,4,4,4-Hexafluoro-2-methyl-but-2-ene | 3,3,4,4,5,5,6,6,6-Nonafluoro-hex-1-ene |
| | 31 | 58-59.5 |
| nBp (° C.) | 3,3,4,5,5,5-Hexafluoro-pent-1-ene | 1,1,3,3,5,5,6,6,6-Nonafluoro-hex-1-ene |
| | 46-47 | 75 |
| nBp (° C.) | 4,4,4-Trifluoro-2-trifluoromethyl-but-1-ene | 4,4,4-Trifluoro-3,3-bis-trifluoromethyl-but-1-ene |
| | 47-49 | 53-58 |
| nBp (° C.) | 1,1,1-Trifluoro-2-trifluoromethyl-but-2-ene | (Z)-1,4,4,5,5,5-Hexafluoro-2-trifluoromethyl-pent-1-ene |
| | | 87 |
| nBp (° C.) | (Z)-1,1,1,4,4,4-Hexafluoro-2-methyl-but-2-ene | (E)-1,4,4,5,5,5-Hexafluoro-2-trifluoromethyl-pent-1-ene |
| | | 71 |
| nBp (° C.) | (E)-1,1,1,4,4,4-Hexafluoro-2-methyl-but-2-ene | 1,1,1-Trifluor-2,3-bis(trifluormethyl)-2-buten |
| | | 34 |
| nBp (° C.) | 4,4,4-Trifluoro-3-trifluoromethyl-but-1-ene | (E)-1,1,1,5,5,5-Hexafluoro-4-trifluoromethyl-pent-2-ene |
| | | 68-70 |
| nBp (° C.) | | (Z)-1,1,1,2,5,5,6,6,6-Nonafluoro-hex-2-ene |

These novel compounds are: (E)-1,1,1,2,4,4,4-heptafluoro-but-2-ene, (E)-1,1,1,2,4,4,4-heptafluoro-but-2-ene, (E)-1,1,1,2,4,4-hexafluoro-but-2-ene, (Z)-1,1,1,2,4,4-hexafluoro-but-2-ene, (E)-1,1,1,3,4,4-hexafluoro-but-2-ene, (Z)-1,1,1,3,4,4-hexafluoro-but-2-ene, (Z)-1,2,3,4,4,4-hexafluoro-but-1-ene, (E)-1,1,1,2,3,4-hexafluoro-but-2-ene, (Z)-1,1,1,2,3,4-hexafluoro-but-2-ene, (Z)-1,2,3,4,4,4-hexafluoro-but-1-ene, (E)-1,1,1,2,3,4-hexafluoro-but-2-ene, (Z)-1,1,1,2,3,4-hexafluoro-but-2-ene, (E)-1,2,4,4,4-Pentafluoro-but-1-ene, (Z)-1,2,4,4,4-Pentafluoro-but-1-ene, (E)-1,1,1,3,4-Pentafluoro-but-2-ene, (Z)-1,1,1,3,4-Pentafluoro-but-2-ene ; 2,3,4,4,4-pentafluoro-but-1-ene, (E)-1,1,1,3-tetrafluoro-but-2-ene, (Z)-1,1,1,3-tetrafluoro-but-2-ene, 2,4,4,4-tetrafluoro-but-2-ene, (E)-1,1,1,2,4,4,5,5,5-nonafluoro-pent-2-ene, (E)-1,1,1,2,3,4,5,5,5-nonafluoro-pent-2-ene, (Z)-1,1,1,2,3,4,5,5,5-nonafluoro-pent-2-ene, (E)-1,1,1,2,3,5,5,5-octafluoro-pent-2-ene, (Z)-1,1,1,2,3,5,5,5-octafluoro-pent-2-ene, (E)-1,1,1,3,4,5,5,5-octafluoro-pent-2-ene, (Z)-1,1,1,3,4,5,5,5-octafluoro-pent-2-ene, (E)-1,1,1,3,5,5,5-octafluoro-pent-2-ene, (Z)-1,1,1,3,5,5,5-octafluoro-pent-2-ene, (E)-1,1,1,2,4,4-hexafluoro-but-2-ene, (Z)-1,1,1,2,4,4-hexafluoro-but-2-ene, (E)-1,1,1,2,2,4-hexafluoro-but-2-ene, (Z)-1,1,1,2,2,4-hexafluoro-but-2-ene, 2,4,4,5,5,5-hexafluoro-but-1-ene, (E)-1,1,1,2,4,4,6,6,6-Nonafluoro-hex-2-ene, (Z)-1,1,1,2,4,4,6,6,6-Nonafluoro-hex-2-ene, (E)-1,1,1,2,2,4,6,6,6-Nonafluoro-hex-3-ene, (Z)-1,1,1,2,2,4,6,6,6-Nonafluoro-hex-3-ene, (E)-1,1,1,3,5,5,6,6,6-Nonafluoro-hex-2-ene and (Z)-1,1,1,3,5,5,6,6,6-Nonafluoro-hex-2-ene.

Further compounds considered novel are (E)-1,2,3,3-Tetrafluoro-propene, 2,3,3,3-Tetrafluoro-propene, 1,1,3,3-Tetrafluoro-propene, (Z)-1,2,3,3-Tetrafluoro-propene, (E)-1,3,3,3-Tetrafluoro-propene, 1,3,3,3-Tetrafluoro-2-trifluoromethyl-propene, 1,1,2,3,3,4,4-Heptafluoro-but-1-ene, 1,1,3,3,4,4,4-Heptafluoro-but-1-ene, (Z)-1,1,1,2,3,4,4-Heptafluoro-but-2-ene, (Z)-1,1,1,4,4,4-Hexafluoro-but-2-ene, (E)-1,1,1,4,4,4-Hexafluoro-but-2-ene, (E)-1,1,1,4,4,4-Hexafluoro-but-2-ene, 3,3,3-Trifluoro-2-trifluoromethyl-propene, (E)-1,1,2,3,4,4-Hexafluoro-but-2-ene, (Z)-1,1,2,3,4,4-Hexafluoro-but-2-ene, (E)-1,1,2,3,4,4-Hexafluoro-but-2-ene, (Z)-1,2,3,3,4,4-Hexafluoro-but-1-ene, 1,1,2,3,3-Pentafluoro-but-1-ene, 1,1,4,4,4-Pentafluoro-but-1-ene, 3,3,4,4,4-Pentafluoro-but-1-ene, 1,1,3,3,3-Pentafluoro-2-methyl-propene, (E)-1,1,2,4,4-Pentafluoro-but-2-ene, 2-Difluoromethyl-3,3,3-trifluoro-propene, (E)-1,1,2,3,4-Pentafluoro-but-2-ene, (Z)-1,1,2,4,4-Pentafluoro-but-2-ene, (Z)-1,2,3,3,4-Pentafluoro-but-1-ene, (Z)-1,1,1,2,4-Pentafluoro-but-2-ene, 1,1,3,3-Tetrafluoro-2-methyl-propene, 3,3,4,4-Tetrafluoro-but-1-ene, 2-Difluoromethyl-3,3-difluoro-propene, (E)-1,1,1,2-Tetrafluoro-but-2-ene, (Z)-1,1,1,2-Tetrafluoro-but-2-ene, (Z)-1,3,3,3-Tetrafluoro-2-methyl-propene, (E)-1,3,3,3-Tetrafluoro-2-methyl-propene, (E)-1,3,3,3-Tetrafluoro-2-methyl-propene, 1,1,4,4-tetrafluoro-1-butene, 1,1,2,3,3,4,4,5,5,5-Decafluoro-pent-1-ene, 1,1,2,3,4,4,4-Heptafluoro-3-trifluoromethyl-but-1-ene, 1,1,1,2,4,4,4-Heptafluoro-3-trifluoromethyl-but-2-ene, 1,1,3,3,4,4,4-Heptafluoro-2-trifluoromethyl-but-1-ene, 1,1,3,3,4,4,5,5,5-Nonafluoro-pent-1-ene, 1,1,3,4,4,4-Hexafluoro-3-trifluoromethyl-but-1-ene, 1,1,2,3,3,4,4,5,5-Nonafluoro-pent-1-ene, (E)-1,2,3,3,4,4,5,5,5-Nonafluoro-pent-1-ene, (Z)-1,1,1,2,4,4,5,5,5-Nonafluoro-pent-2-ene, (Z)-1,2,3,3,4,4,5,5,5-Nonafluoro-pent-1-ene, (Z)-1,1,1,2,3,4,4,5,5-Nonafluoro-pent-2-ene, (E)-1,1,1,2,3,4,4,5,5-Nonafluoro-pent-2-ene, (Z)-1,1,2,3,4,4,5,5,5-Nonafluoro-pent-2-ene, (E)-1,1,2,3,4,4,5,5,5-Nonafluoro-pent-2-ene, 1,1,4,4,4-Pentafluoro-2-trifluoromethyl-but-1-ene, (Z)-1,3,4,4,4-Pentafluoro-3-trifluoromethyl-but-1-ene, (E)-1,3,4,4,4-Pentafluoro-3-trifluoromethyl-but-1-ene, (Z)-1,3,4,4,4-Pentafluoro-3-trifluoromethyl-but-1-ene, (Z)-1,3,3,4,4,5,5,5-Octafluoro-pent-1-ene, (E)-1,3,3,4,4,5,5,5-Octafluoro-pent-1-ene, 3,3,4,4,4-Pentafluoro-2-trifluoromethyl-but-1-ene, 3,3,4,4,5,5,5-Heptafluoro-pent-1-ene, 1,1,1,3-Tetrafluoro-2-trifluoromethyl-but-2-ene, 2,3,3,4,4,5,5-Heptafluoro-pent-1-ene, 1,1,3,3,5,5,5-Heptafluoro-pent-1-ene, (E)-1,1,1,2,4,4,4-Heptafluoro-3-methyl-but-2-ene, (Z)-1,1,1,2,4,4,4-Heptafluoro-3-methyl-but-2-ene, 3,4,4,4-Tetrafluoro-3-trifluoromethyl-but-1-ene, (E)-1,1,1,4,4,4-Hexafluoro-2-methyl-but-2-ene, 3,3,4,5,5,5-Hexafluoro-pent-1-ene, 4,4,4-Trifluoro-2-trifluoromethyl-but-1-ene, 1,1,1-Trifluoro-2-trifluoromethyl-but-2-ene, (Z)-1,1,1,4,4,4-Hexafluoro-2-methyl-but-2-ene, (E)-1,1,1,4,4,4-Hexafluoro-2-methyl-but-2-ene, 4,4,4-Trifluoro-3-trifluoromethyl-but-1-ene, 3,3,4,4,5,5,6,6,6-Nonafluoro-hex-1-ene, 1,1,3,3,5,5,6,6,6-Nonafluoro-hex-1-ene, 4,4,4-Trifluoro-3,3-bis-trifluoromethyl-but-1-ene, (Z)-1,4,4,5,5,5-Hexafluoro-2-trifluoromethyl-pent-1-ene, (E)-1,4,4,5,5,5-Hexafluoro-2-trifluoromethyl-pent-1-ene, 1,1,1-Trifluor-2,3-bis(trifluormethyl)-2-butene, (E)-1,1,1,5,5,5-Hexafluoro-4-trifluoromethyl-pent-2-ene and (Z)-1,1,1,2,5,5,6,6,6-Nonafluoro-hex-2-ene.

Of the novel compounds, preferred ones are those having at least 1 hydrogen atom and equal to or more than 6 fluorine atoms. Especially preferred compounds are (E)-CF₃—CH═CF—CH₂—CF₃ and (Z)—CF₃—CH═CF—CH₂—CF₃.

The invention also concerns a method for transferring of heat, for drying a solid surface of an article using a solvent or for degreasing parts using a solvent wherein the hydrofluoroalkenes obtainable according to the present invention are applied. Hydrofluoroalkenes having at least 1 hydrogen atom and equal to or more than 6 fluorine atoms are preferred. The hydrofluoroalkenes and mixtures thereof can be applied together with
other heat transfer fluids, for example, partially fluorinated or perfluorinated polyethers, e. g. a perfluoropolyether of formula (I), CF₃—[(OCF(CF₃)—CF₂)ₐ—(O—CF₂)_{b}]O—CF₃ (I), wherein said perfluoropolyether has a boiling point of about 57° C. at 101.3 kPa and an average molecular mass of about 340, available as Galden® HT55, or a perfluoropolyether having a boiling point of about 66° C. at 101.3 kPa at a pressure of about 101.3 kPa, available as Galden® HT70, both from Solvay Solexis, or perfluorinated ketones, for example, perfluoroethyl-perfluoroisopropyl ketone,
other drying agents or degreasing agents, e.g. an alkane, alkene, or an alcohol in proportions as mentioned above. For example, it can be applied together with trans-dichloroethylene or an alcohol, for example, methanol, ethanol or isopropanol, and a stabilizer in the proportions mentioned above.

Especially preferred is a method for transferring of heat, for drying a solid surface of an article using a solvent or for degreasing parts using a solvent wherein (E)-CF₃—CH═CF—CH₂—CF₃ and (Z)—CF₃—CH═CF—CH₂—CF₃ and mixtures thereof is used as a heat-transfer fluid, as a drying solvent or as a degreasing solvent. As mentioned above, these compounds can be applied together with other heat-transfer fluids, drying solvents or degreasing solvents.

Another subject of the present invention is a composition of matter comprising a hydrofluorolefin obtainable according to the process of the present invention and at least one other component. Preferably, this other component is a compound suitable as blowing agent or as additive of blowing agents; a compound suitable as heat transfer fluid, or a compound suitable as solvent for drying or degreasing purposes. Preferred compositions comprise (E)-CF₃—CH═CF—CH₂—CF₃ and (Z)—CF₃—CH═CF—CH₂—CF₃ and mixtures thereof.

Blowing agents, especially alkanes, e.g. propane, n-butane, iso-butane, pentane, cyclopropane, cyclobutane, cyclopentane, alkenes, hydrofluoroalkanes, e.g. difluoromethane, tetrafluoroethane, pentafluoropropane, hexafluoropropane, heptafluoropropane, hydrofluoroalkenes, e.g. those with 2 to 5 carbon atoms, alcohols, e.g. methanol, or carbon dioxide are suitable as compounds in blowing agent compositions containing the hydrofluoroalkenes obtainable according to the present invention.

The other compound can also be selected from blowing agent additives, especially from the group consisting of polyester polyols, polyether polyols, and flame retardants, e.g. phosphate esters or phosphonate esters.

The at least one other component in the composition of matter may be a heat transfer fluid, for example, a partially fluorinated or perfluorinated polyether, e. g. a perfluoropolyether of formula (I), CF₃—[(OCF(CF₃)—CF₂)ₐ—(O—CF₂)_{b}]O—CF₃ (I), wherein said perfluoropolyether has a boiling point of about 57° C. at 101.3 kPa and an average molecular mass of about 340, available as Galden® HT55, or a perfluoropolyether having a boiling point of about 66° C. at 101.3 kPa at a pressure of about 101,3 kPa, available as Galden® HT70, both from Solvay Solexis, or a perfluorinated ketone, for example, perfluoroethyl-perfluoroisopropyl ketone,

The at least one other component in the compositions of the present invention may be a drying agent or degreasing agent, for example an alkane, alkene, e.g. dichloroethylene, or an alcohol in proportions as mentioned above. For example, the composition according to the invention comprises (E)-CF₃—CH═CF—CH₂—CF₃ and (Z)—CF₃—CH═CF—CH₂—CF₃ and mixtures thereof, trans-dichloroethylene or an alcohol, for example, methanol, ethanol or isopropanol, and optionally a stabilizer in the proportions mentioned above.

The following examples explain the invention in more detail without intending to limit it.

### EXAMPLE 1

### Preparation of a chloroalkene

A mixture which contains approximately 56% by weight of 3-chloro-1,1,3-tetrafluorobutane, 10% by weight of 1,1-dichloro-1,3,3-trifluorobutane, 7% by weight of 1,1-difluoro-1,1,3-trichlorobutane and 4% by weight 1-1,1,3,3-tetrafluorobutane and other halogenated C4 compounds is obtained from the non-catalytic liquid phase reaction of 1,1,1,3,3-pentachlorobutane and HF. High surface AlF₃, prepared and activated as described in WO 2009/010472, is introduced into a fixed bed reactor. The starting material was passed as vapor in a nitrogen stream through the catalyst bed. The dehydrofluorination reaction was performed at a temperature of 200° C. The resulting gas stream was passed over NaF to remove HF and condensed. The condensed liquid was analyzed by GC-MS and NMR. The typical product distribution of the resulting reaction mixture is compiled in the following table:

TABLE
Dehydrofluorination products

| Retention | GC-Area | |
|---|---|---|
| time [min] | [%] | Products |
| 6.5 | 1.9 | HFC-365 |
| 10.1 | 5.6 | HFO-1353 |
| 10.4 | 10.1 | HFC-364 |
| 10.9 | 3.2 | HFC-364 (other isomer) |
| 11.0 | 54.1 | HFO-1353 |
| 11.9 | 16.0 | HFO-1353 |
| | 9.2 | Rest |

The table shows that the 3 isomers of CH₄ClF₃ have a retention time of 10.1 minutes, 11.0 minutes and 11.9 minutes. Especially NMR analysis revealed that the isomer with a retention time of 11.0 minutes is 2-chloro-3,3,3-trifluorobutene. Consequently, the conversion of HFC-364 was greater than 90%.

### EXAMPLE 2

### Synthesis of 1,1,1,3,3-pentachloro-5,5,5-trifluoropentane

The raw product of example 1 was used as starting material without further isolation. It was reacted with an excess of CCl₄ which had the function of reactant and solvent. The telomerization reaction was performed overnight in the presence of CuCl₂ and tert-butyl amine at about 100 to 110° C. A 90% conversion of 2-chloro-3,3,3-trifluorobutene to form 1,1,1,3,3-pentachloro-5,5,5-trifluoropentane was observed.

### EXAMPLE 3

### Telomerization with Purified 2-chloro-3,3,3-trifluorobutene

From a raw product of fluoro and chlorofluorobutenes as obtained in example 1,2-chloro-3,3,3-trifluorobutene was isolated by distillation. The telomerization reaction was performed as in example 2. According to the GC analysis, the conversion of 2-chloro-3,3,3-trifluorobutene was about 90%, and the yield of 1,1,1,3,3-pentachloro-5,5,5-trifluoropentane was more than 80%.

### EXAMPLE 4

### Synthesis of 1,1,1,3,3,5,5,5-octafluoropentane

1700 g of 1,1,1,3,3-pentachloro-5,5,5-trifluoropentane, 1140 g HF and 300 g SbCl₅ were introduced into a 5-1 reaction vessel. The molar ratios were 1.0:10.0:0.18, respectively (the stoichiometrical molar ratio of HF to alkane is 5:1). After feeding the HF to the reactor, the pressure increased to 11 to 12 bar at room temperature at first. Then, the temperature was increased step by step to 70° C. HCl was continuously purged from the reactor at the indicated pressure. The reaction mixture was kept for several hours under these conditions. After cooling the reactor, two main fractions were observed. The weight of the depressurized reaction mixture was 1220 g. After washing it with water, an organic fraction of 1100 g remained. The analytical GC data are compiled in the following table.

TABLE
GC data of the raw fluorination product

| Retention | GC- Area | |
|---|---|---|
| time [min] | [%] | Products |
| 7.9 | 92.8 | HFC-458 |
| 14.0 | 4.8 | Unknown* |
| 29.8 | 0.0 | HFC-453 |
| | 2.4 | Rest |

*Already observed in the starting material

The organic fraction was distilled under 450 mbar, the top and bottom temperatures were 42.8 and 46.7° C. 936 g of HFC-458 were obtained with a purity of 99%.

### EXAMPLE 5

### Synthesis of 1,1,1,3,5,5,5-heptafluoro-2-pentene

The synthesis of 1,1,1,3,5,5,5-heptafluoro-2-pentene from the 1,1,1,3,3,5,5,5-octafluoropentane of example 4 by dehydrofluorination was carried out in a lab-scale tubular flow reactor filled with 0.8 g of the high surface aluminium catalyst which also was used in example 1. The inner diameter of the reactor was 5 mm. HFC-458 was carried as vapor in a nitrogen stream through the catalyst bed. The reaction was performed at a temperature of 330° C. The gases leaving the reactor were passed through a NaF tower and analyzed via GC.

The analytical data are compiled in the following table.

TABLE
GC data of the raw dehyfluorination product

| Retention | GC- Area | |
|---|---|---|
| time [min] | [%] | Products |
| 7.2 | 35.1 | HFO-1447 |
| 7.8 | 13.4 | HFC-458 |
| 9.5 | 45.9 | HFO-1447 |
| | 5.6 | Rest |

The data show that the E and Z isomers of 1,1,1,3,5,5,5-heptafluoro-2-pentene are obtained in roughly the same amounts. To improve the yield, unreacted HFC-458 could be returned to the dehydrofluorination reaction after its isolation.

### EXAMPLE 6

### Blowing Agent Compositions and Their Use

90 g of a polyetherpolyol (Tercarol A350) is mixed with 10 g of a mixture of the isomers of the hydrofluoroolefin HFO-1447 (E/Z-1,1,1,3,5,5,5-heptafluoro-2-pentene) as obtained in example 5. Then, 20 g of triethylphosphate is added as flame retardant.

The resulting premix is then reacted with 2,6-toluene diisocyanate in the presence of dimethyl cyclohexylamine as catalyst to form a foamed polyurethane.

### EXAMPLE 7

### Drying Agent Compositions

100 g of the HFO-1447 composition of example 5 are mixed with 35 g of trans-dichloroethylene and 1.5 g isopropanol. The mixture is suitable for degreasing metal parts and as drying agent, e.g. for drying moist metal parts.

1. A process for the manufacture of a hydroolefin, comprising the steps of
a) providing a chlorinated precursor compound
b) fluorinating said chlorinated precursor compound to provide a fluorinated precursor compound
c) eliminating HF from said fluorinated precursor compound to form at least one hydrofluoroolefin.

2. The process of claim 1 wherein the chlorinated precursor compound is provided by a reaction of a chlorinated alkene with a chlorine-containing compound.

3. The process of claim 2 wherein the chlorine-containing compound is selected from the group consisting of Cl₂, CCl₄ and CCl₃-CCl₃.

4. The process of claim 3 wherein the reaction with chlorine is photochemically assisted, and wherein the reaction with CCl₄ and CCl₃-CCl₃ is catalyzed by Cu(I) or Cu(II) compounds.

5. (canceled)

6. (canceled)

7. The process of claim 1 wherein the chlorinated alkene is one of formula (I),
R¹CH═CClR² (I)
wherein R¹ is H; a C₁ to C₃ alkyl group; or a C₁ to C₃ alkyl group which is substituted by at least 1 halogen atom selected from the group consisting of chlorine and fluorine; and
wherein R² is H; a C₁ to C₃ alkyl group; or a C₁ to C₃ alkyl group which is substituted by at least 1 halogen atom selected from the group consisting of chlorine and fluorine.

8. The process of claim 7 wherein the chlorinated alkene is selected from the group consisting of CH₂═CHCl, CH₂═CCl—CH₃, CH₂═C(Cl)—CCl₃ and CH₂═C(Cl)CH₂—CF₃.

9. The process of claim 1 wherein the fluorinated precursor compound is
one of formula (IIIa), (IIIb) or (IIIc)
R¹CH₂—CF₂—R² (IIIa)
R¹CHF—CF₂—R² (IIIb)
R¹CF₂—CF₂—R² (IIIc)
wherein R¹ is H; F; a C₁ to C₃ alkyl group; or a C₁ to C₃ alkyl group which is substituted by at least 1 fluorine atom; and wherein R² is H; a C₁ to C₃ alkyl group; or a C₁ to C₃ alkyl group which is substituted by at least 1 fluorine atom,
with the proviso that the number of carbon atoms in the fluorinated precursor compounds of formulae (IIIa), (IIIb) and (IIIc) is an integer equal to or greater than 3, and the number of fluorine atoms is at least 4.

10. The process of claim 9 wherein R¹ is selected from the group consisting of F; CF₃; CF₃CH₂; CF₃CHF; and CF₃CF₂; and wherein R² is selected from the group consisting of H; CH₃; CH₂F; CHF₂; CF₃CH₂; CF₃CHF; and CF₃CF₂.

11. The process of claim 1 wherein the hydrofluoroolefin is one of formula (IV).
CₐH_{b}F_{c} (IV)
wherein a, b and c are integers, a is from 4 to 8, b is from 4 to 10 and c is (2a-b), and a+b+c are 2a.

12. The process of claim 11 wherein a is from 4 to 6, b is from 1 to 4, and c is (2a-b).

13. (canceled)

14. The process of claim 11 wherein the hydrofluoroolefin is selected from the group consisting of C₃H₂F₄, C₄H₄F₄, C₄H₃F₅, C₄H₂F₆, CₒH₄F₆, C₄H₁F₇, C₅H₃F₇, C₅H₂F₈, C₅H₁F₉ and C₆H₃F₉.

15. The process of claim 14 wherein the hydrofluoroolefin is selected from the group consisting of (E)-CF₃—CH═CF—CH₂—CF₃, (Z)—CF₃—CH═CF—CH₂—CF₃, and mixtures thereof.

16. A method for foam blowing, for transferring of heat, for drying a solid surface of an article using a solvent or for degreasing parts using a solvent, comprising using a hydrofluoroolefin obtainable according to the process of claim 1 as a blowing agent, as a heat-transfer fluid, as a drying solvent or as a degreasing solvent.

17. A composition of matter comprising a hydrofluoroolefin obtainable according to the process of claim 1, and a blowing agent or blowing agent additive, a heat transfer fluid, or a solvent.

18. A hydrofluoroolefin or any isomer thereof selected from the group consisting of (E)-1,1,1,2,4,4,4-heptafluoro-but-2-ene, (E)-1,1,1,2,4,4,4-heptafluoro-but-2-ene, (E)-1,1,1,2,4,4-hexafluoro-but-2-ene, (Z)-1,1,1,2,4,4-hexafluoro-but-2-ene, (E)-1,1,1,3,4,4-hexafluoro-but-2-ene, (Z)-1,1,1,3,4,4-hexafluoro-but-2-ene, (Z)-1,2,3,4,4,4-hexafluoro-but-1-ene, (E)-1,1,1,2,3,4-hexafluoro-but-2-ene, (Z)-1,1,1,2,3,4-hexafluoro-but-2-ene, (Z)-1,2,3,4,4,4-hexafluoro-but-1-ene, (E)-1,1,1,2,3,4-hexafluoro-but-2-ene, (Z)-1,1,1,2,3,4-hexafluoro-but-2-ene, (E)-1,2,4,4,4-Pentafluoro-but-1-ene, (Z)-1,2,4,4,4-Pentafluoro-but-1-ene, (E)-1,1,1,3,4-Pentafluoro-but-2-ene, (Z)-1,1,1,3,4-Pentafluoro-but-2-ene; 2,3,4,4,4-pentafluoro-but-1-ene, (E)-1,1,1,3-tetrafluoro-but-2-ene, (Z)-1,1,1,3-tetrafluoro-but-2-ene, 2,4,4,4-tetrafluoro-but-2-ene, (E)-1,1,1,2,4,4,5,5,5-nonafluoro-pent-2-ene, (E)-1,1,1,2,3,4,5,5,5-nonafluoro-pent-2-ene, (Z)-1,1,1,2,3,4,5,5,5-nonafluoro-pent-2-ene, (E)-1,1,1,2,3,5,5,5-octafluoro-pent-2-ene, (Z)-1,1,1,2,3,5,5,5-octafluoro-pent-2-ene, (E)-1,1,1,3,4,5,5,5-octafluoro-pent-2-ene, (Z)-1,1,1,3,4,5,5,5-octafluoro-pent-2-ene, (E)-1,1,1,3,5,5,5-octafluoro-pent-2-ene, (Z)-1,1,1,3,5,5,5-octafluoro-pent-2-ene, (E)-1,1,1,2,4,4-hexafluoro-but-2-ene, (Z)-1,1,1,2,4,4-hexafluoro-but-2-ene, (E)-1,1,1,2,2,4-hexafluoro-but-2-ene, (Z)-1,1,1,2,2,4-hexafluoro-but-2-ene, 2,4,4,5,5,5-hexafluoro-but-1-ene, (E)-1,1,1,2,4,4,6,6,6-Nonafluoro-hex-2-ene, (Z)-1,1,1,2,4,4,6,6,6-Nonafluoro-hex-2-ene, (E)-1,1,1,2,2,4,6,6,6-Nonafluoro-hex-3-ene, (Z)-1,1,1,2,2,4,6,6,6-Nonafluoro-hex-3-ene, (E)-1,1,1,3,5,5,6,6,6-Nonafluoro-hex-2-ene and (Z)-1,1,1,3,5,5,6,6,6-Nonafluoro-hex-2-ene, (E)-1,2,3,3-Tetrafluoro-propene, 2,3,3,3-Tetrafluoro-propene, 1,1,3,3-Tetrafluoro-propene, (Z)-1,2,3,3-Tetrafluoro-propene, (E)-1,3,3,3-Tetrafluoro-propene, 1,3,3,3-Tetrafluoro-2-trifluoromethyl-propene, 1,1,2,3,3,4,4-Heptafluoro-but-1-ene, 1,1,3,3,4,4,4-Heptafluoro-but-1-ene, (Z)-1,1,1,2,3,4,4-Heptafluoro-but-2-ene, (Z)-1,1,1,4,4,4-Hexafluoro-but-2-ene, (E)-1,1,1,4,4,4-Hexafluoro-but-2-ene, (E)-1,1,1,4,4,4-Hexafluoro-but-2-ene, 3,3,3-Trifluoro-2-trifluoromethyl-propene, (E)-1,1,2,3,4,4-Hexafluoro-but-2-ene, (Z)-1,1,2,3,4,4-Hexafluoro-but-2-ene, (E)-1,1,2,3,4,4-Hexafluoro-but-2-ene, (Z)-1,2,3,3,4,4-Hexafluoro-but-1-ene, 1,1,2,3,3-Pentafluoro-but-1-ene, 1,1,4,4,4-Pentafluoro-but-1-ene, 3,3,4,4,4-Pentafluoro-but-1-ene, 1,1,3,3,3-Pentafluoro-2-methyl-propene, (E)-1,1,2,4,4-Pentafluoro-but-2-ene, 2-Difluoromethyl-3,3,3-trifluoro-propene, (E)-1,1,2,3,4-Pentafluoro-but-2-ene, (Z)-1,1,2,4,4-Pentafluoro-but-2-ene, (Z)-1,2,3,3,4-Pentafluoro-but-1-ene, (Z)-1,1,1,2,4-Pentafluoro-but-2-ene, 1,1,3,3-Tetrafluoro-2-methyl-propene, 3,3,4,4-Tetrafluoro-but-1-ene, 2-Difluoromethyl-3,3-difluoro-propene, (E)-1,1,1,2-Tetrafluoro-but-2-ene, (Z)-1,1,1,2-Tetrafluoro-but-2-ene, (Z)-1,3,3,3-Tetrafluoro-2-methyl-propene, (E)-1,3,3,3-Tetrafluoro-2-methyl-propene, (E)-1,3,3,3-Tetrafluoro-2-methyl-propene, 1,1,4,4-tetrafluoro-1-butene, 1,1,2,3,3,4,4,5,5,5-Decafluoro-pent-1-ene, 1,1,2,3,4,4,4-Heptafluoro-3-trifluoromethyl-but-1-ene, 1,1,1,2,4,4,4-Heptafluoro-3-trifluoromethyl-but-2-ene, 1,1,3,3,4,4,4-Heptafluoro-2-trifluoromethyl-but-1-ene, 1,1,3,3,4,4,5,5,5-Nonafluoro-pent-1-ene, 1,1,3,4,4,4-Hexafluoro-3-trifluoromethyl-but-1-ene, 1,1,2,3,3,4,4,5,5-Nonafluoro-pent-1-ene, (E)-1,2,3,3,4,4,5,5,5-Nonafluoro-pent-1-ene, (Z)-1,1,1,2,4,4,5,5,5-Nonafluoro-pent-2-ene, (Z)-1,2,3,3,4,4,5,5,5-Nonafluoro-pent-1-ene, (Z)-1,1,1,2,3,4,4,5,5-Nonafluoro-pent-2-ene, (E)-1,1,1,2,3,4,4,5,5-Nonafluoro-pent-2-ene, (Z)-1,1,2,3,4,4,5,5,5-Nonafluoro-pent-2-ene, (E)-1,1,2,3,4,4,5,5,5-Nonafluoro-pent-2-ene, 1,1,4,4,4-Pentafluoro-2-trifluoromethyl-but-1-ene, (Z)-1,3,4,4,4-Pentafluoro-3-trifluoromethyl-but-1-ene, (E)-1,3,4,4,4-Pentafluoro-3-trifluoromethyl-but-1-ene, (Z)-1,3,4,4,4-Pentafluoro-3-trifluoromethyl-but-1-ene, (Z)-1,3,3,4,4,5,5,5-Octafluoro-pent-1-ene, (E)-1,3,3,4,4,5,5,5-Octafluoro-pent-1-ene, 3,3,4,4,4-Pentafluoro-2-trifluoromethyl-but-1-ene, 3,3,4,4,5,5,5-Heptafluoro-pent-1-ene, 1,1,1,3-Tetrafluoro-2-trifluoromethyl-but-2-ene, 2,3,3,4,4,5,5-Heptafluoro-pent-1-ene, 1,1,3,3,5,5,5-Heptafluoro-pent-1-ene, (E)-1,1,1,2,4,4,4-Heptafluoro-3-methyl-but-2-ene, (Z)-1,1,1,2,4,4,4-Heptafluoro-3-methyl-but-2-ene, 3,4,4,4-Tetrafluoro-3-trifluoromethyl-but-1-ene, (E)-1,1,1,4,4,4-Hexafluoro-2-methyl-but-2-ene, 3,3,4,5,5,5-Hexafluoro-pent-1-ene, 4,4,4-Trifluoro-2-trifluoromethyl-but-1-ene, 1,1,1-Trifluoro-2-trifluoromethyl-but-2-ene, (Z)-1,1,1,4,4,4-Hexafluoro-2-methyl-but-2-ene, (E)-1,1,1,4,4,4-Hexafluoro-2-methyl-but-2-ene, 4,4,4-Trifluoro-3-trifluoromethyl-but-1-ene, 3,3,4,4,5,5,6,6,6-Nonafluoro-hex-1-ene, 1,1,3,3,5,5,6,6,6-Nonafluoro-hex-1-ene, 4,4,4-Trifluoro-3,3-bis-trifluoromethyl-but-1-ene, (Z)-1,4,4,5,5,5-Hexafluoro-2-trifluoromethyl-pent-1-ene, (E)-1,4,4,5,5,5-Hexafluoro-2-trifluoromethyl-pent-1-ene, 1,1,1-Trifluor-2,3-bis(trifluormethyl)-2-butene, (E)-1,1,1,5,5,5-Hexafluoro-4-trifluoromethyl-pent-2-ene, and (Z)-1,1,1,2,5,5,6,6,6-Nonafluoro-hex-2-ene.

19. The method of claim 16 wherein the hydrofluoroolefin is selected from (E)-CF₃—CH═CF—CH₂—CF₃, (Z)—CF₃—CH═CF—CH₂—CF₃, and mixtures thereof.

20. The composition of claim 17 wherein the hydrofluoroolefin is selected from (E)-CF₃—CH═CF—CH₂—CF₃, (Z)—CF₃—CH═CF—CH₂—CF₃, and mixtures thereof.
